(19) United States
(12) Patent Application Publication
(10) Pub. No.: US 2026/0131015 A1
(43) Pub. Date: May 14, 2026

(54) NEODEGRADER CONJUGATES

(71) Applicant: Orum Therapeutics, Inc., Yuseong-gu (KR)

(72) Inventors: Nathan FISHKIN, Cambridge, MA (US); Min-Soo KIM, Daejeon (KR); Khuloud TAKROURI, Wayland, MA (US); James PALACINO, Wellesley, MA (US); Dong-Ki CHOI, Daejeon (KR); Da-Yeong KIM, Daejeon (KR); Hangyeol JEONG, Daejeon (KR)

(21) Appl. No.: 19/118,644

(22) PCT Filed: Oct. 6, 2023
(86) PCT No.: PCT/IB2023/060064

§ 371 Date: Apr. 4, 2025

Related U.S. Application Data
(60) Provisional application No. 63/378,663, filed on Oct. 6, 2022.

(51) Int. Cl. A61K 47/68; A61K 45/06; A61P 35/00; C07K 16/28

(52) U.S. Cl. CPC A61K 47/6803; A61K 45/06; A61K 47/6849; A61K 47/6889; A61P 35/00; C07K 16/2803

(57) ABSTRACT

The present disclosure provides neoDegraders conjugated to binding moieties that specifically bind to CD56. Also provided are compositions comprising the neoDegrader conjugates. The compounds and compositions are useful for treating a disease or condition, e.g., cancer, in a subject in need thereof.

### CROSS-REFERENCE TO RELATED APPLICATIONS

This application claims the priority benefit of U.S. Provisional Application No. 63/378,663, filed Oct. 6, 2022, which is incorporated herein by reference in its entirety.

### REFERENCE TO SEQUENCE LISTING SUBMITTED ELECTRONICALLY

The content of the electronically submitted sequence listing (Name 4547_026PC01_Seqlisting_ST26; Size: 25,060 bytes; and Date of Creation: Oct. 4, 2023), filed with the application is incorporated herein by reference in its entirety.

### FIELD

The present disclosure provides neoDegrader conjugates, wherein the neoDegrader is conjugated to a binding moiety that specifically binds to CD56. Also provided are compositions comprising the conjugates. The conjugates and compositions are useful for treating cancer in a subject in need thereof.

### BACKGROUND

Protein degradation has been validated as a therapeutic strategy by the effectiveness of immunomodulatory imide drugs. These compounds have the ability to bind to cereblon (CRBN) and promote recruitment and ubiquitination of substrate proteins mediated by CRL4^{CRBN} E3 ubiquitin ligase. It is thought that immunomodulatory imides act as “molecular glues,” filling the binding interface as a hydrophobic patch that reprograms protein interactions between the ligase and neosubstrates.

Despite the excitement for these compounds as novel treatments for cancer, thus far they have been limited to use in hematologic malignancies such as multiple myeloma and myelodysplastic syndrome (MDS). Expanding the library of compounds that can function by degrading other oncoproteins, many of which have been considered ‘undruggable,’ is an active area of drug development. Thus there is a continuing need for new compounds that can target these alternative oncoproteins and treat a wide array of cancers.

### SUMMARY

In some aspects, the present disclosure provides a conjugate of formula (I) or formula (II):

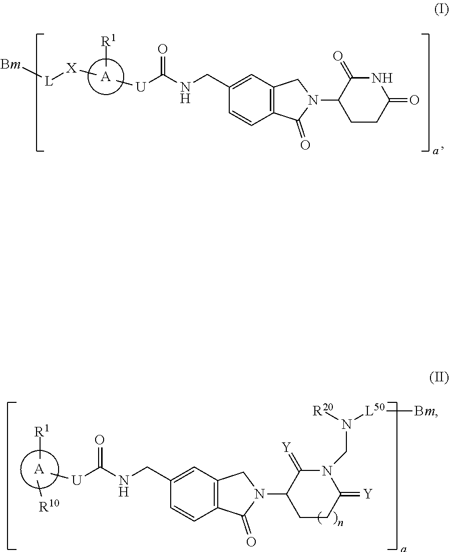

or a pharmaceutically acceptable salt thereof, wherein:
a is an integer from 1 to 10;
n is 0 or 1;
A is phenyl or a C₄-C₁₀cycloalkyl ring;
U is selected from NH, O, S, and CF₂;
R¹ is independently selected from hydrogen and halo;
R¹⁰ is selected from —CH₃, —C(O)R³⁰, —N(R⁴⁰)₂, —(CH₂)_{n′}OH, —(CH₂)_{n′}N(R⁴⁰)₂, —(CH₂)_{n′}Q(CH₂)_{m′}OH, —(CH₂)_{n′}Q(CH₂)_{m′}SH, and —(CH₂)_{n′}Q(CH₂)_{m′}N(R⁴⁰)₂; wherein:
   R³⁰ is hydrogen or C₁-C₆alkyl;
   each R⁴⁰ is independently hydrogen or C₁-C₆alkyl;
   Q is O, S, or NR⁴⁰;
   n′ is 1-6; and
   m′ is 2-5;
R²⁰ is selected from hydrogen, —(CH₂CH₂O)_{v′}—CH₃, C₂-C₆alkenyl, C₁-C₆alkyl; C₂-C₆alkynyl, benzyl, C₃-C₆cycloalkyl, and C₃-C₆cycloalkyl(C₁-C₃alkyl), wherein v′ is from 1 to 24;
X is selected from —NR²⁰⁰—, =C(CH₃)—, -Q′—(CH₂)_{n′}—, and -Q′(CH₂)_{m″}Q″(CH₂)_{n″}—; wherein:
   Q′ and Q″ are each independently O, S, or N(R²⁰⁰)ᵥ, wherein:
      v is 1 or 2;
      each R²⁰⁰ is independently hydrogen or C₁-C₆alkyl;
   n″ is an integer from 1 to 6; and
   m″ is an integer from 2 to 6;
   wherein the left side of each group is attached to L and the right side is attached to A;
provided that when X is NH or -Q′—(CH₂)_{n′}—, R¹ is halo;
each Y is independently S or O;
L is a cleavable linker or non-cleavable linker;
L⁵⁰ is a cleavable linker;
and
Bm is a binding moiety that is capable of specifically binding to CD56.

In some aspects, the binding moiety is an antibody or an antigen-binding fragment thereof.

In some aspects, a is an integer from 2 to 8.

In some aspects, L is a non-cleavable linker.

In some aspects, L is selected from the group consisting of

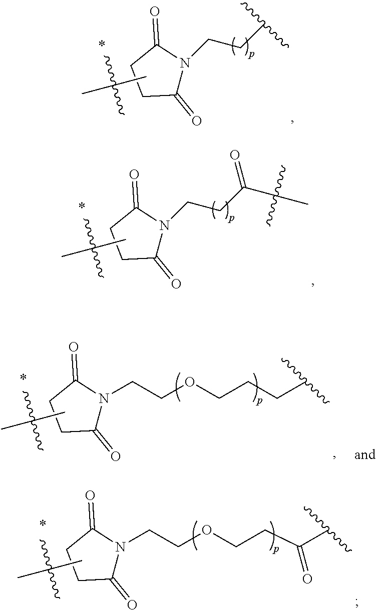

wherein:
   p is an integer from 1 to 10;
[symbol] is the point of attachment to X; and
   [symbol] is the point of attachment to the binding moiety.

In some aspects, L is

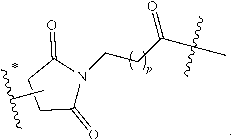

In some aspects, p is 5.

In some aspects, the conjugate is a conjugate of formula (I) or a pharmaceutically acceptable salt thereof, wherein L is a cleavable linker. In some aspects, the cleavable linker is cleavable by a protease. In some aspects, L is selected from the group consisting of

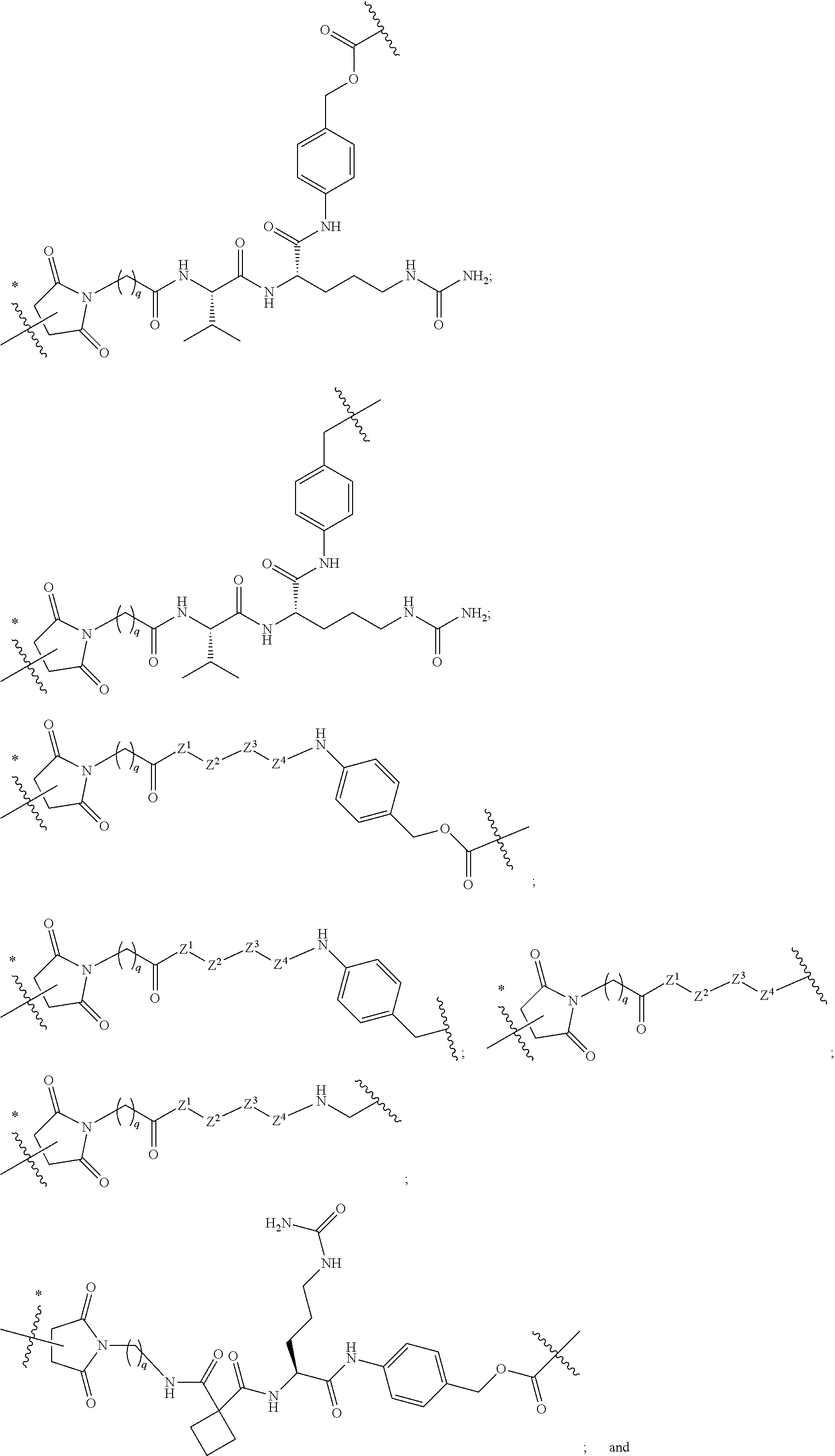

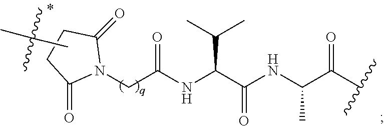

q is an integer from 2 to 10;
Z¹, Z², Z³, and Z⁴ are each independently absent or a naturally-occurring amino acid residue in the L- or D-configuration, provided that at least two of Z¹, Z², Z³, and Z⁴ are amino acid residues;
[symbol] is the point of attachment to X; and
[symbol] is the point of attachment to the binding moiety.

In some aspects, Z¹, Z², Z³, and Z⁴ are independently absent or selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, and Z⁴ are amino acid residues.

In some aspects, Z¹ is absent or glycine; Z² is absent or selected from the group consisting of L-glutamine, D-glutamine, L-glutamic acid, D-glutamic acid, L-aspartic acid, D-aspartic acid, L-alanine, D-alanine, and glycine; Z³ is selected from the group consisting of L-valine, D-valine, L-alanine, D-alanine, L-phenylalanine, D-phenylalanine, and glycine; and Z⁴ is selected from the group consisting of L-alanine, D-alanine, L-citrulline, D-citrulline, L-asparagine, D-asparagine, L-lysine, D-lysine, L-phenylalanine, D-phenylalanine, and glycine.

In some aspects, L is

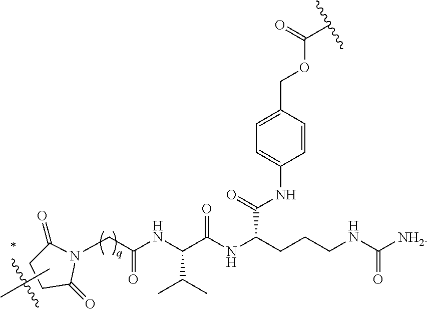

In some aspects, q is 5.

In some aspects, L is a bioreducible linker.

In some aspects, L is selected from the group consisting of

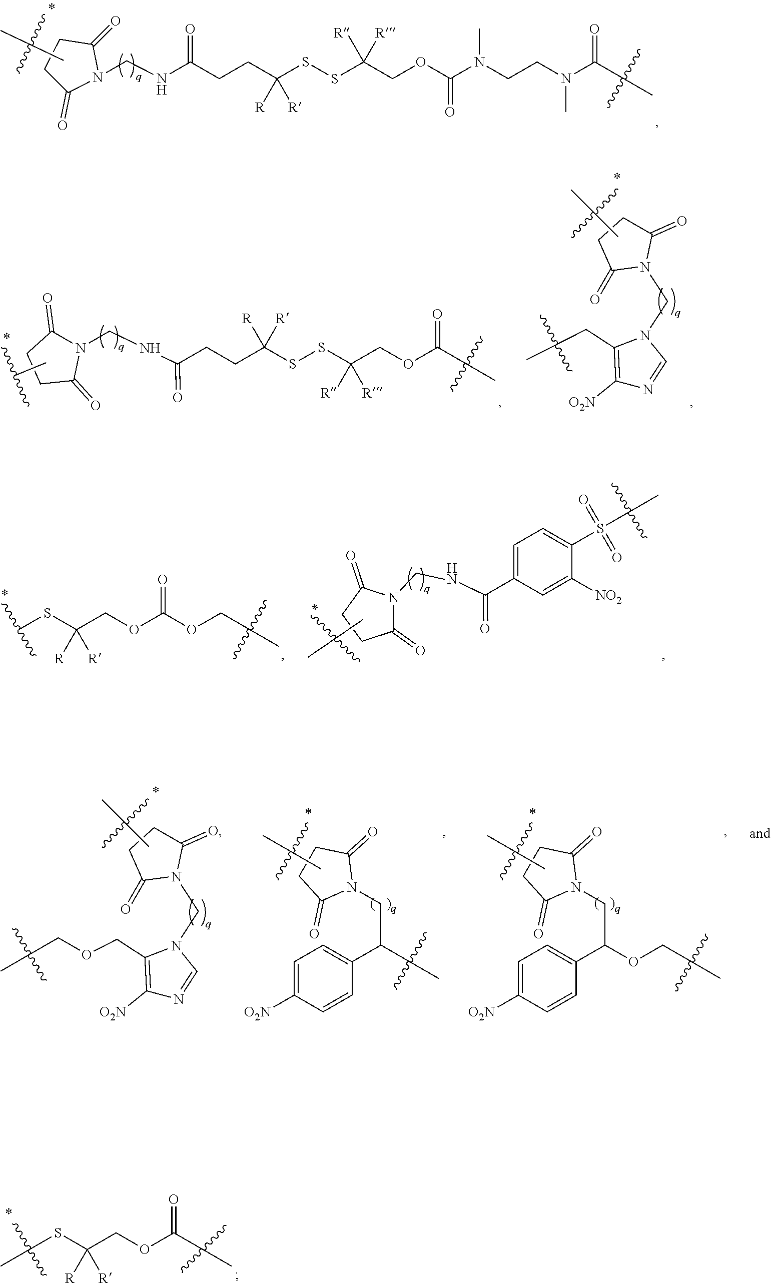

wherein:
   q is an integer from 2 to 10;
   R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring;
   [symbol] is the point of attachment to X; and
   [symbol] is the point of attachment to the binding moiety.

In some aspects, L is an acid cleavable linker.

In some aspects, L is selected from the group consisting of

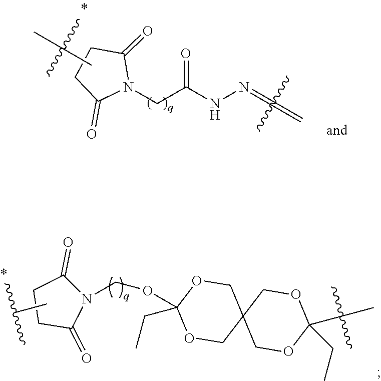

wherein:
   q is an integer from 2 to 10;
   [symbol] is the point of attachment to X; and
   [symbol] is the point of attachment to the binding moiety.

In some aspects, L is a click-to-release linker. In some aspects, L is selected from

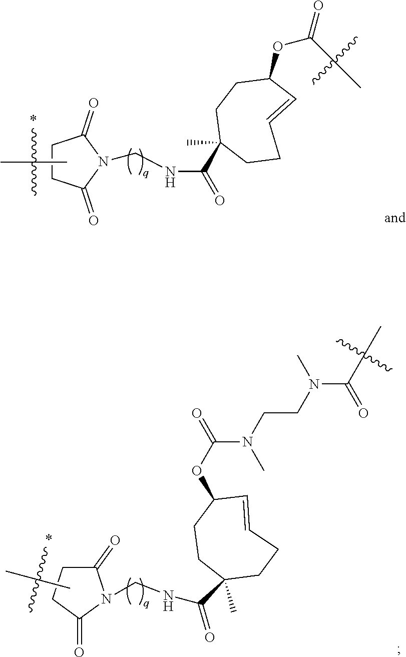

wherein:
   q is an integer from 2 to 10;
   [symbol] is the point of attachment to X; and
   [symbol] is the point of attachment to the binding moiety.

In some aspects, L is a pyrophosphatase cleavable linker. In some aspects, L is

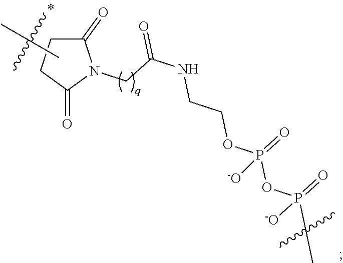

wherein:
   q is an integer from 2 to 10;
   [symbol] is the point of attachment to X; and
   [symbol] is the point of attachment to the binding moiety.

In some aspects, L is a beta-glucuronidase cleavable linker. In some aspects, L is selected from

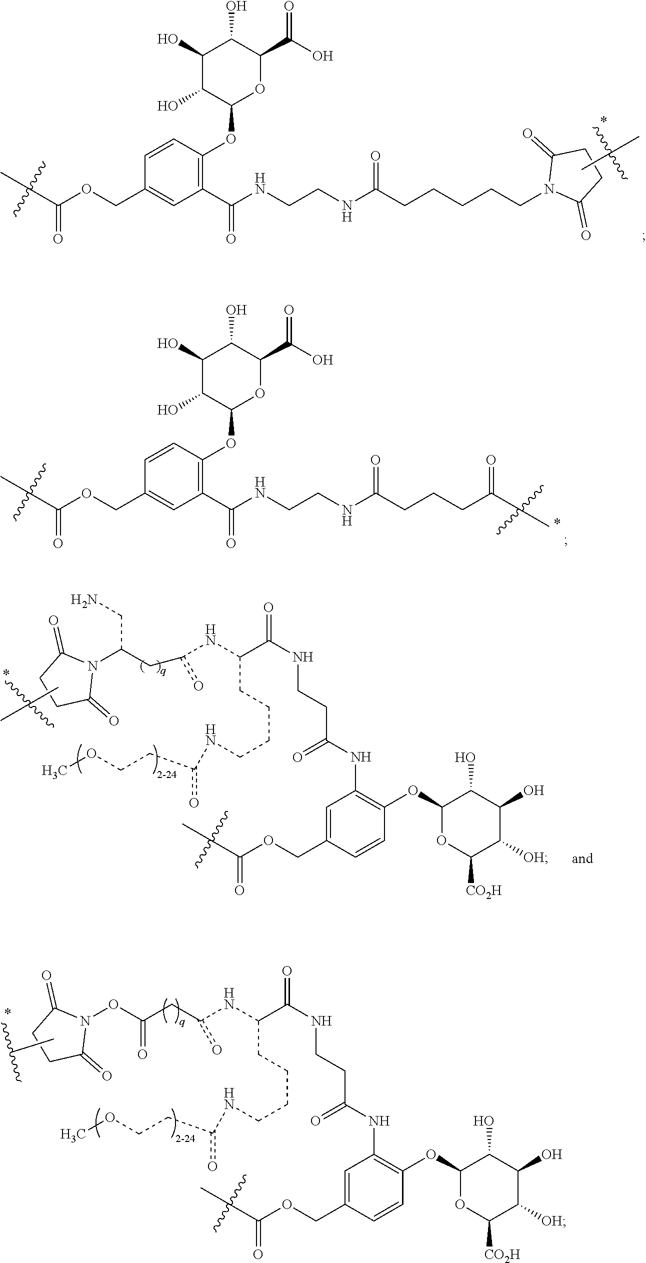

wherein:
   q is an integer from 2 to 10;
   [symbol] is absent or a bond;
   [symbol] is the point of attachment to X; and
   [symbol] is the point of attachment to the binding moiety.

In some aspects, L is

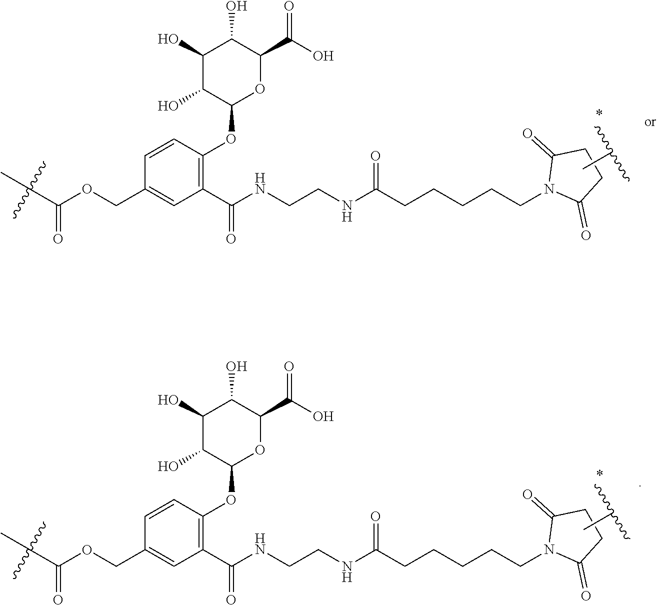

In some aspects, the conjugate is a conjugate of formula (I), or a pharmaceutically acceptable salt thereof, wherein:
A is phenyl;
U is NH;
R¹ is halo; and
X is —N(R²⁰⁰)ᵥ(CH₂)_{m″}O(CH₂)_{n″}—; wherein:
   v is 1;
   m″ and n″ are 2; and
   R²⁰⁰ is methyl.

In some aspects, the conjugate is a conjugate of formula (I), or a pharmaceutically acceptable salt thereof, wherein:
A is phenyl;
U is NH;
R¹ is halo; and
X is —N(R²⁰⁰)ᵥ(CH₂)_{m″}O(CH₂)_{n″}—; wherein:
   v is 2;
   m″ and n″ are 2; and
   each R²⁰⁰ is methyl.

In some aspects, the conjugate is a conjugate of formula (I), or a pharmaceutically acceptable salt thereof, wherein:
A is phenyl;
U is NH;
R¹ is halo; and
X is —O(CH₂)_{n″}—; wherein:
   n″ is 2.

In some aspects, the conjugate is a conjugate of formula (I), or a pharmaceutically acceptable salt thereof, wherein:
A is phenyl;
U is NH;
R¹ is halo; and
X is —S(CH₂)_{n″}—; wherein:
   n″ is 2.

In some aspects, the conjugate is a conjugate of formula (I), or a pharmaceutically acceptable salt thereof, wherein:
A is phenyl;
U is NH;
R¹ is hydrogen; and
X is —NR²⁰⁰—; wherein:
   R²⁰⁰ is methyl.

In some aspects, the conjugate is a conjugate of formula (I), or a pharmaceutically acceptable salt thereof, wherein:
A is phenyl;
U is NH;
R¹ is halo; and
X is —NR²⁰⁰—; wherein:
   R²⁰⁰ is hydrogen.

In some aspects, the conjugate is a conjugate of formula (I), or a pharmaceutically acceptable salt thereof, wherein:
A is phenyl;
U is NH;
R¹ is halo; and
X is —NR²—; wherein:
   R² is hydrogen.

In some aspects, the conjugate is a conjugate of formula (I), or a pharmaceutically acceptable salt thereof, wherein:
A is phenyl;
U is NH;
R¹ is hydrogen; and
X is —C(CH₃)═.

In some aspects, the conjugate is a conjugate of formula (I), or a pharmaceutically acceptable salt thereof, wherein:
A is a C₄-C₁₀cycloalkyl ring;
U is NH;
R¹ is hydrogen; and
X is —N(R²⁰⁰)(CH₂)ₘO(CH₂)ₙ—; wherein:
   n″ is 1;
   m″ is 2; and
   R²⁰⁰ is methyl.

In some aspects, the conjugate is a conjugate of formula (II), or a pharmaceutically acceptable salt thereof, wherein the cleavable linker is cleavable by a protease. In some aspects, L⁵⁰ is selected from the group consisting of

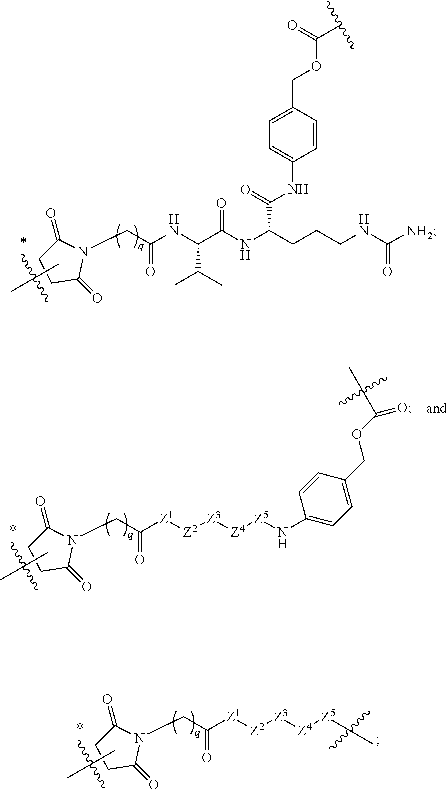

wherein:
   q is from 2 to 10;
   Z¹, Z², Z³, Z⁴, and Z⁵ are each independently absent or a naturally-occurring amino acid residue in the L- or D-configuration, provided that at least two of Z¹, Z², Z³, Z⁴, and Z⁵ are amino acid residues;
   [symbol] is the point of attachment to the parent molecular moiety; and
   [symbol] is the point of attachment to the binding moiety.

In some aspects, Z¹, Z², Z³, Z⁴, and Z⁵ are independently absent or selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, Z⁴, and Z⁵ are amino acid residues.

In some aspects,
Z¹ is absent or glycine;
Z² is absent or selected from the group consisting of L-glutamine, D-glutamine, L-glutamic acid, D-glutamic acid, L-aspartic acid, D-aspartic acid, L-alanine, D-alanine, and glycine;
Z³ is selected from the group consisting of L-valine, D-valine, L-alanine, D-alanine, L-phenylalanine, D-phenylalanine, and glycine;
Z⁴ is selected from the group consisting of L-citrulline, D-citrulline, L-asparagine, D-asparagine, L-lysine, D-lysine, L-phenylalanine, D-phenylalanine, and glycine; and
Z⁵ is absent or glycine.

In some aspects, L⁵⁰ is

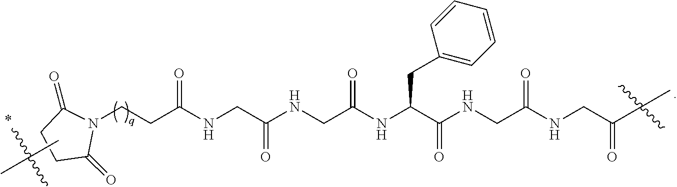

In some aspects, q is 4.

In some aspects, the conjugate is a conjugate of formula (II), or a pharmaceutically acceptable salt thereof, wherein L⁵⁰ is a bioreducible linker. In some aspects, L⁵⁰ is selected from the group consisting of

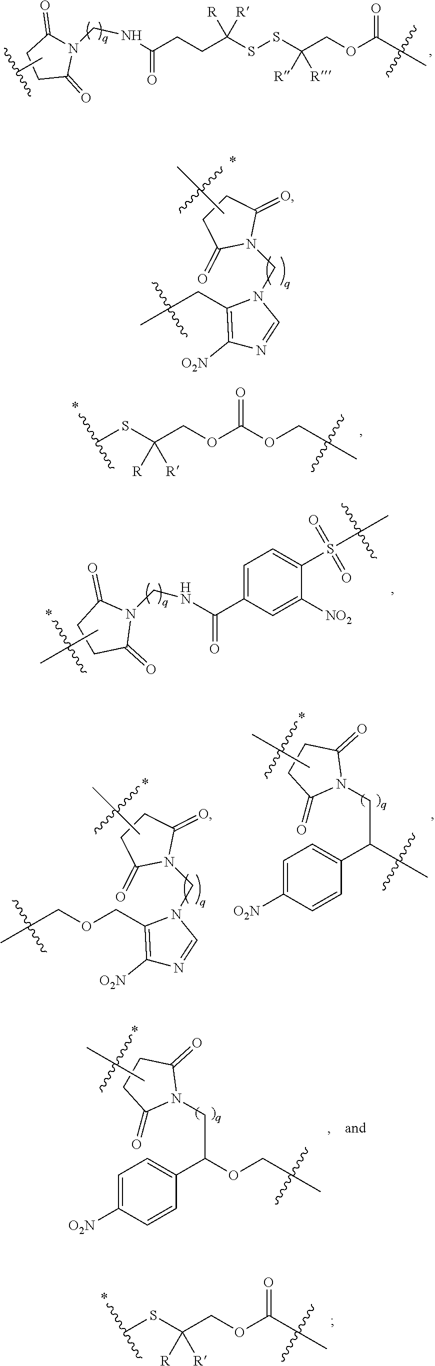

wherein:
   q is from 2 to 10;
   R, R′, R″, and R′″ are each independently selected from hydrogen, C₁-C₆alkoxyC₁-C₆alkyl, (C₁-C₆alkyl)₂NC₁-C₆alkyl, and C₁-C₆alkyl, or, two geminal R groups, together with the carbon atom to which they are attached, can form a cyclobutyl or cyclopropyl ring;
   [symbol] is the point of attachment to the parent molecular moiety; and
   [symbol] is the point of attachment to the binding moiety.

In some aspects, L⁵⁰ is

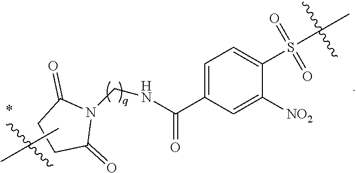

In some aspects q is 2.

In some aspects, the conjugate is a conjugate of formula (II), or a pharmaceutically acceptable salt thereof, wherein L⁵⁰ is a click-to-release linker. In some aspects, L⁵⁰ is

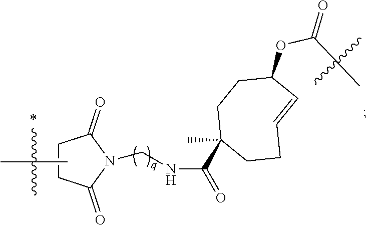

wherein:
   q is from 2 to 10;
   [symbol] is the point of attachment to the parent molecular moiety; and
   [symbol] is the point of attachment to the binding moiety.

In some aspects, the conjugate is a conjugate of formula (II), or a pharmaceutically acceptable salt thereof, wherein L⁵⁰ is a beta-glucuronidase cleavable linker. In some aspects, L⁵⁰ is selected from

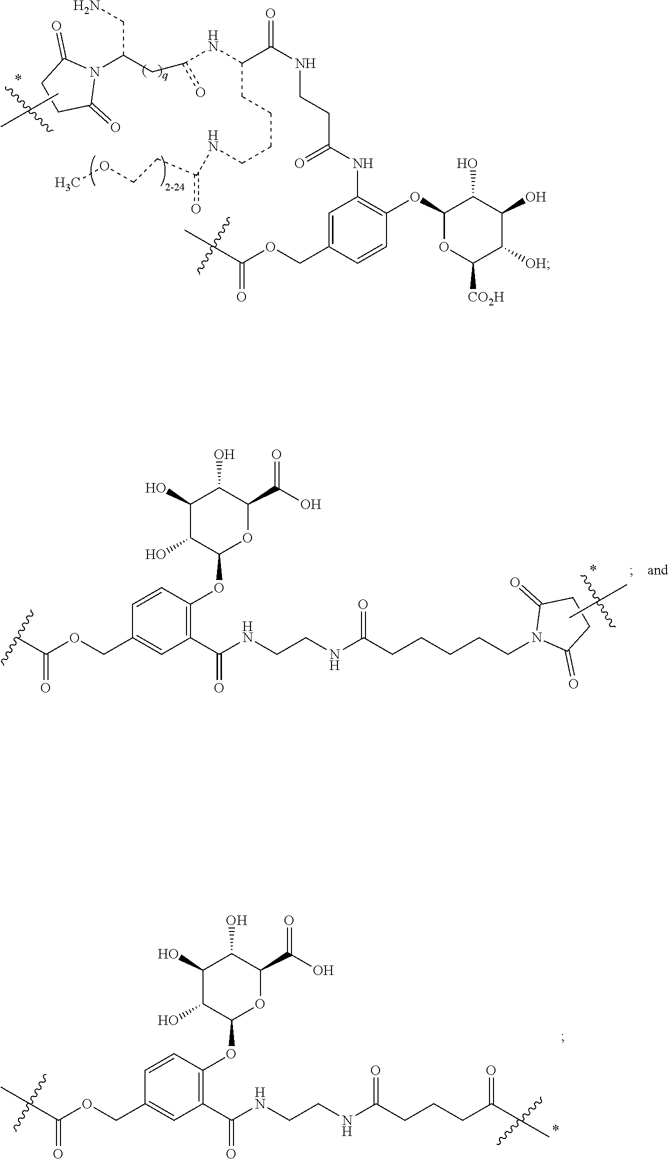

wherein:
   q is from 2 to 10;
   [symbol] is absent or a bond;
   [symbol] is the point of attachment to the parent molecular moiety; and
   [symbol] is the point of attachment to the binding moiety.

In some aspects, a is from 3 to 4.

In some aspects, a is 8.

In some aspects, the binding moiety comprises a variable heavy chain (VH) complementarity determining region (CDR) 1 comprising the amino acid sequence of SEQ ID NO:2, a VH CDR2 comprising the amino acid sequence of SEQ ID NO:3, a VH CDR3 comprising the amino acid sequence of SEQ ID NO:4, a variable light chain (VL) CDR1 comprising the amino acid sequence of SEQ ID NO:5, a VL CDR2 comprising the amino acid sequence of SEQ ID NO:6, and a VL CDR3 comprising the amino acid sequence of SEQ ID NO:7. In some aspects, the binding moiety comprises a VH comprising the amino acid sequence of SEQ ID NO:8 and/or a VL comprising the amino acid sequence of SEQ ID NO:9. In some aspects, the antibody or antigen-binding fragment thereof is an IgG antibody or an antigen-binding fragment thereof. In some aspects, the IgG antibody or antigen-binding fragment thereof is an IgG1 antibody or antigen-binding fragment thereof. In some aspects, the antibody or antigen-binding fragment thereof comprises (i) an N297A mutation according to EU numbering and/or (ii) a LALA (L234A and L235A) mutation according to EU numbering. In some aspects, the antibody or antigen-binding fragment thereof comprises a constant region comprising an engineered cysteine or a constant region comprising the amino acid sequence of any one of SEQ ID NOs:14-20. In some aspects the binding moiety comprises (i) a heavy chain comprising the amino acid sequence of SEQ ID NO:10 and/or a light chain comprising the amino acid sequence of SEQ ID NO:11 or (ii) a heavy chain comprising the amino acid sequence of SEQ ID NO:12 and/or a light chain comprising the amino acid sequence of SEQ ID NO:11.

In some aspects, the present disclosure provides a conjugate which is:

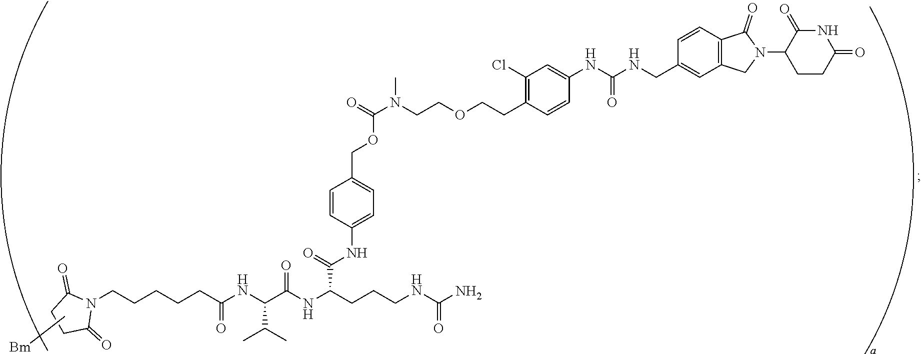

wherein Bm is antibody CD56-B and a is 3-8. In some aspects a is 3, 4, or 8.

In some aspects, the present disclosure provides a composition comprising at least one conjugate provided herein, wherein the average number of neodegraders per Bm is 3-8. In some aspects, the average number of neodegraders per Bm is about 3 to about 4. In some aspects, the average number of neodegraders per Bm is about 8.

In some aspects, the present disclosure provides a method of treating cancer in a subject in need thereof, the method comprising administering to the subject a pharmaceutically acceptable amount of a conjugate or composition provided herein, or a pharmaceutically acceptable salt thereof. In some aspects, the cancer is CD56-positive cancer. In some aspects, the cancer is a neuroendocrine cancer (optionally wherein the neuroendocrine cancer is a neuroblastoma or neuroendocrine prostate cancer), a lung cancer (optionally wherein the lung cancer is a small cell lung cancer (SLCL)), or a sarcoma. In some aspects, the method further comprises administering to the subject a pharmaceutically acceptable amount of an additional agent prior to, after, or simultaneously with the conjugate or composition provided herein or a pharmaceutically acceptable salt thereof. In some aspects, the additional agent is a cytotoxic agent or an immune response modifier. In some aspects, the immune response modifier is a checkpoint inhibitor. In some aspects, the checkpoint inhibitor comprises a PD-1 inhibitor, a PD-L1 inhibitor, a CTLA-4 inhibitor, a TIM3 inhibitor, and/or a LAG-3 inhibitor.

In some aspects, the present disclosure provides a use of a conjugate or composition provided herein in the preparation of a medicament for a use provided herein.

### BRIEF DESCRIPTION OF THE FIGURES

FIG. 1 depicts a native SEC-MS graph showing the distribution of Compound (III)—antibody CD56-A conjugate.

FIG. 2 depicts a reducing LC-MS of antibody CD56-A (light chain and heavy chain) compared to the Compound (III)—antibody CD56-A conjugate.

FIG. 3 depicts a native SEC-MS graph showing the distribution of conjugated species for Compound (III)—antibody CD56-B conjugate.

FIG. 4 depicts a reducing LC-MS of antibody CD56-B (light chain and heavy chain) compared with Compound (III)—antibody CD56-B conjugate.

FIG. 5A depicts the activity of Conjugate (VIII) and the Compound (IV)—antibody CD56-B DAR8 conjugate against NCI-H660 cell line.

FIG. 5B depicts individual tumor volume overtime for each dose group in Example 10.

FIG. 5C depicts average body weight for mice in each dose group over course of the study described in Example 10.

FIG. 6A depicts the activity of Conjugate (VI) against NCI-H1048 cell line.

FIG. 6B depicts individual tumor volume overtime for each dose group in Example 11.

FIG. 6C depicts average body weight for mice in each dose group over course of the study described in Example 11.

FIG. 7 depicts a native SEC-MS graph showing the distribution of conjugated species for Compound (IV-1)—antibody CD56-B conjugate.

FIG. 8 depicts a native SEC-MS graph showing the distribution of conjugated species for Compound (IV)—antibody CD56-B conjugate.

FIG. 9A depicts the activity of Conjugate (IX) against IMR32 human neuroblastoma cancer tumor cells.

FIG. 9B depicts individual tumor volume overtime for each dose group in Example 12.

FIG. 9C depicts average body weight for mice in each dose group over course of the study described in Example 12.

### DETAILED DESCRIPTION

The present disclosure is directed to a conjugate of formula (I) or formula (II):

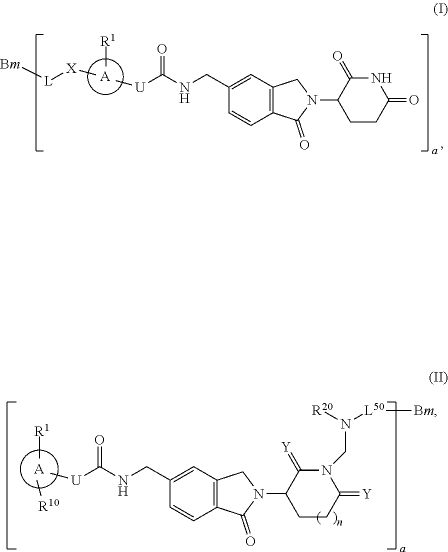

or a pharmaceutically acceptable salt thereof, wherein:
a is an integer from 1 to 10;
n is 0 or 1;
A is phenyl or a C₄-C₁₀cycloalkyl ring;
U is selected from NH, O, S, and CF₂;
R¹ is independently selected from hydrogen and halo;
R¹⁰ is selected from —CH₃, —C(O)R³⁰, —N(R⁴⁰)₂, —(CH₂)_{n′}OH, —(CH₂)_{n′}N(R⁴⁰)₂, —(CH₂)_{n′}Q(CH₂)_{m′}OH, —(CH₂)_{n′}Q(CH₂)_{m′}SH, and —(CH₂)_{n′}Q(CH₂)_{m′}N(R⁴⁰)₂; wherein:
   R³⁰ is hydrogen or C₁-C₆alkyl;
   each R⁴⁰ is independently hydrogen or C₁-C₆alkyl;
   Q is O, S, or NR⁴⁰;
   n′ is 1-6; and
   m′ is 2-5;
   R²⁰ is selected from hydrogen, —(CH₂CH₂O)_{v′}—CH₃, C₂-C₆alkenyl, C₁-C₆alkyl; C₂-C₆alkynyl, benzyl, C₃-C₆cycloalkyl, and C₃-C₆cycloalkyl(C₁-C₃alkyl), wherein v′ is from 1 to 24;
X is selected from —NR²⁰⁰—, =C(CH₃)—, -Q′—(CH₂)_{n′}—, and -Q′(CH₂)_{m″}Q″(CH₂)_{n″}—; wherein:
   Q′ and Q″ are each independently O, S, or N(R²⁰⁰)ᵥ, wherein:
      v is 1 or 2; and
      each R²⁰⁰ is independently hydrogen or C₁-C₆alkyl;
   n″ is an integer from 1 to 6; and
   m″ is an integer from 2 to 6;
wherein the left side of each group is attached to L and the right side is attached to A;
provided that when X is NH or -Q′—(CH₂)_{n″}—, R¹ is halo;
each Y is independently S or O;
L is a cleavable linker or non-cleavable linker;
L⁵⁰ is a cleavable linker;
and
Bm is a binding moiety that is capable of specifically binding to CD56. In some aspects, the binding moiety is an antibody or an antigen-binding fragment.

The present disclosure also provides compositions comprising the conjugates, and the method of using and making the conjugates.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The terms “targeted protein degrader,” and “neoDegrader,” as used herein, refer to a molecule that forms a ternary complex with an E3 ubiquitin ligase which is capable of targeting a protein for degradation. Examples include, but are not limited to, molecular glues and PROTACs. Examples of molecular glues include, but are not limited to CC-90009, lenalidomide, pomalidomide, mezigdomide (CC-92480), iberdomide (CC-220), DKY709, and Compound P1 disclosed in WO2021/198965.

The term “DAR,” as used herein, refers to the drug antibody ratio of conjugates, which is the average number of neoDegrader-linker complexes linked to each binding moiety (e.g., antibody or antigen-binding fragment thereof). In certain aspects, the DAR of the conjugates described herein is from 1 to 10. In some aspects, the DAR of the conjugates described herein is from 1 to 8. In some aspects, the DAR of the conjugates described herein is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.

The term “antibody,” as used herein, also refers to a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cells. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

An “intact antibody” is one which comprises an antigen-binding variable region as well as a light chain constant domain (CL) and heavy chain constant domains, CH1, CH2 and CH3. The constant domains may be native sequence constant domains (e.g., human native sequence constant domains) or amino acid sequence variant thereof.

“Antibody fragments” comprise a portion of an intact antibody, generally the antigen binding or variable region thereof. Examples of antibody fragments include Fab, Fab′, F(ab′)₂, and Fv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “single domain antibody,” also known as a nanobody, is an antibody fragment consisting of a single monomeric variable antibody domain with a molecular weight of from about 12 kDa to about 15 kDa. Single body antibodies can be based on heavy chain variable domains or light chains. Examples of single domain antibodies include, but are not limited to, V_{H}H fragments and V_{NAR} fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method, or may be made by recombinant DNA methods. The “monoclonal antibodies” may also be isolated from phage antibody libraries.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies of interest herein include “primatized” antibodies comprising variable domain antigen-binding sequences derived from a non-human primate (e.g., Old World Monkey, Ape etc.) and human constant region sequences.

Various methods have been employed to produce monoclonal antibodies (MAbs). Hybridoma technology, which refers to a cloned cell line that produces a single type of antibody, uses the cells of various species, including mice (murine), hamsters, rats, and humans. Another method to prepare MAbs uses genetic engineering including recombinant DNA techniques. Monoclonal antibodies made from these techniques include, among others, chimeric antibodies and humanized antibodies. A chimeric antibody combines DNA encoding regions from more than one type of species. For example, a chimeric antibody may derive the variable region from a mouse and the constant region from a human. A humanized antibody comes predominantly from a human, even though it contains nonhuman portions. Like a chimeric antibody, a humanized antibody may contain a completely human constant region. But unlike a chimeric antibody, the variable region may be partially derived from a human. The nonhuman, synthetic portions of a humanized antibody often come from CDRs in murine antibodies. In any event, these regions are crucial to allow the antibody to recognize and bind to a specific antigen. While useful for diagnostics and short-term therapies, murine antibodies cannot be administered to people long-term without increasing the risk of a deleterious immunogenic response. This response, called Human Anti-Mouse Antibody (HAMA), occurs when a human immune system recognizes the murine antibody as foreign and attacks it. A HAMA response can cause toxic shock or even death.

Chimeric and humanized antibodies reduce the likelihood of a HAMA response by minimizing the nonhuman portions of administered antibodies. Furthermore, chimeric and humanized antibodies can have the additional benefit of activating secondary human immune responses, such as antibody dependent cellular cytotoxicity.

The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g., B cell receptor; BCR), etc.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The terms “administration,” “administering,” and grammatical variants thereof refer to introducing a composition, such as an EV (e.g., exosome) of the present disclosure, into a subject via a pharmaceutically acceptable route. The introduction of a composition, such as an EV (e.g., exosome) of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least about 70% identical, at least about 80% identical, at least about 90% identical, or at least about 95% identical to one another. In some aspects, two or more sequences are said to be “conserved” if they are at least about 30% identical, at least about 40% identical, at least about 50% identical, at least about 60% identical, at least about 70% identical, at least about 80% identical, at least about 90% identical, or at least about 95% identical to one another. Conservation of sequence can apply to the entire length of a polynucleotide or polypeptide or can apply to a portion, region or feature thereof.

The term “neoDegrader conjugate” as used herein refers to a neoDegrader attached to a binding moiety (e.g., an antibody or antigen-binding fragment thereof) through a linker.

As used herein, the terms “linking” and “conjugating” are used interchangeably an each refer to the covalent or non-covalent attachment of two or more moieties comprising a neoDegrader and a binding moiety (e.g., an antibody or antigen-binding fragment thereof). In some aspects the

The term “amino acid sequence variant” refers to polypeptides having amino acid sequences that differ to some extent from a native sequence polypeptide. Ordinarily, amino acid sequence variants will possess at least about 70% sequence identity with at least one receptor binding domain of a native antibody or with at least one ligand binding domain of a native receptor, and typically, they will be at least about 80%, more typically, at least about 90% homologous by sequence with such receptor or ligand binding domains. The amino acid sequence variants possess substitutions, deletions, and/or insertions at certain positions within the amino acid sequence of the native amino acid sequence. Amino acids are designated by the conventional names, one-letter and three-letter codes.

“Sequence identity” is defined as the percentage of residues in the amino acid sequence variant that are identical after aligning the sequences and introducing gaps, if necessary, to achieve the maximum percent sequence identity. Methods and computer programs for the alignment are well known in the art. One such computer program is “Align 2,” authored by Genentech, Inc., which was filed with user documentation in the United States Copyright Office, Washington, D.C. 20559, on Dec. 10, 1991.

The terms “Fc receptor” or “FcR” are used to describe a receptor that binds to the Fc region of an antibody. An exemplary FcR is a native sequence human FcR. Moreover, a FcR may be one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. Other FcRs, including those to be identified in the future, are encompassed by the term “FcR” herein. The term also includes the neonatal receptor, FcRn, which is responsible for the transfer of maternal IgGs to the fetus.

“Complement dependent cytotoxicity” or “CDC” refers to the ability of a molecule to lyse a target in the presence of complement. The complement activation pathway is initiated by the binding of the first component of the complement system (C1q) to a molecule (e.g., an antibody) complexed with a cognate antigen. To assess complement activation, a CDC assay may be performed.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a 0-sheet configuration, connected by three hypervariable regions, which form loops connecting, and in some cases forming part of, the j-sheet structure. The hypervariable regions in each chain are held together in close proximity by the FRs and, with the hypervariable regions from the other chain, contribute to the formation of the antigen-binding site of antibodies. The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 24-34 (L1), 50-56 (L2) and 89-97 (L3) in the light chain variable domain and 31-35 (H1), 50-65 (H2) and 95-102 (H3) in the heavy chain variable domain; Kabat et al supra) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain). “Framework Region” or “FR” residues are those variable domain residues other than the hypervariable region residues as herein defined.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa and lambda, based on the amino acid sequences of their constant domains.

“Single-chain Fv” or “scFv” antibody fragments comprise the VH and VL domains of antibody, wherein these domains are present in a single polypeptide chain. The Fv polypeptide may further comprise a polypeptide linker between the VH and VL domains which enables the scFv to form the desired structure for antigen binding.

The term “diabodies” refers to small antibody fragments with two antigen-binding sites, which fragments comprise a variable heavy domain (VH) connected to a variable light domain (VL) in the same polypeptide chain (VH-VL). By using a linker that is too short to allow pairing between the two domains on the same chain, the domains are forced to pair with the complementary domains of another chain and create two antigen-binding sites.

“Humanized” forms of non-human (e.g., rodent) antibodies are chimeric antibodies that contain minimal sequence derived from non-human immunoglobulin. Humanization is a method to transfer the murine antigen binding information to a non-immunogenic human antibody acceptor, and has resulted in many therapeutically useful drugs. The method of humanization generally begins by transferring all six murine complementarity determining regions (CDRs) onto a human antibody framework. These CDR-grafted antibodies generally do not retain their original affinity for antigen binding, and in fact, affinity is often severely impaired. Besides the CDRs, select non-human antibody framework residues must also be incorporated to maintain proper CDR conformation. The transfer of key mouse framework residues to the human acceptor in order to support the structural conformation of the grafted CDRs has been shown to restore antigen binding and affinity. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In certain aspects, the antibody will be purified (1) to greater than 95% by weight of antibody as determined by the Lowry method, or more than 99% by weight, (2) to a degree sufficient to obtain at least 15 residues of N-terminal or internal amino acid sequence by use of a gas phase protein sequencer, or (3) to homogeneity by SDS-PAGE under reducing or nonreducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell. As used herein, the term “T cell” and “T lymphocytes” are interchangeable and refer to any lymphocytes produced or processed by the thymus gland. In some aspects, a T cell is a CD4+ T cell. In some aspects, a T cell is a CD8+ T cell. In some aspects, a T cell is a NKT cell.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein.

The term “therapeutically effective amount” or “therapeutically effective dosage” refers to an amount of an agent (e.g., neoDegrader or neoDegrader conjugate disclosed herein) that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the composition can, for example, (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer.

In some aspects, a “therapeutically effective amount” is the amount of the neoDegrader or neoDegrader conjugate clinically proven to affect a significant decrease in cancer or slowing of progression (regression) of cancer, such as an advanced solid tumor. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the term “standard of care” refers to a treatment that is accepted by medical experts as a proper treatment for a certain type of disease and that is widely used by healthcare professionals. The term can be used interchangeable with any of the following terms: “best practice,” “standard medical care,” and “standard therapy.”

By way of example, an “anti-cancer agent” promotes cancer regression in a subject or prevents further tumor growth. In certain aspects, a therapeutically effective amount of the drug promotes cancer regression to the point of eliminating the cancer.

The terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

As used herein, the term “immune checkpoint inhibitor” refers to molecules that totally or partially reduce, inhibit, interfere with or modulate one or more checkpoint proteins. Checkpoint proteins regulate T-cell activation or function. Numerous checkpoint proteins are known, such as CTLA-4 and its ligands CD80 and CD86; and PD-1 with its ligands PD-L1 and PD-L2. Pardoll, D. M., Nat Rev Cancer 12(4):252-64 (2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies.

The terms “treat” or “treatment” refer to both therapeutic treatment and prophylactic or preventative measures, wherein the object is to prevent or slow down (lessen) an undesired physiological change or disorder, such as the development or spread of cancer. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

### II. NeoDegraders

The present disclosure provides conjugates of one or more neoDegraders disclosed herein and a binding moiety.

In some aspects, the neoDegrader degrades “G1 to S phase transition protein 1” homolog (GSPT1). Human GSPT1 has been assigned UniProt accession number P15170. Human GSPT1 can have the amino acid sequence of SEQ ID NO:1.

(SEQ ID NO: 1)
MELSEPIVENGETEMSPEESWEHKEEISEAEPGGGSLGDGRPPEE
SAHEMMEEEEEIPKPKSVVAPPGAPKKEHVNVVFIGHVDAGKSTI
GGQIMYLTGMVDKRTLEKYEREAKEKNRETWYLSWALDTNQEERD
KGKTVEVGRAYFETEKKHFTILDAPGHKSFVPNMIGGASQADLAV
LVISARKGEFETGFEKGGQTREHAMLAKTAGVKHLIVLINKMDDP
TVNWSNERYEECKEKLVPFLKKVGFNPKKDIHFMPCSGLTGANLK
EQSDFCPWYIGLPFIPYLDNLPNFNRSVDGPIRLPIVDKYKDMGT
VVLGKLESGSICKGQQLVMMPNKHNVEVLGILSDDVETDTVAPGE
NLKIRLKGIEEEEILPGFILCDPNNLCHSGRTFDAQIVIIEHKSI
ICPGYNAVLHIHTCIEEVEITALICLVDKKSGEKSKTRPRFVKQD
QVCIARLRTAGTICLETFKDFPQMGRFTLRDEGKTIAIGKVLKLV
PEKD

In some aspects, the present disclosure provides neoDegraders of formula (X):

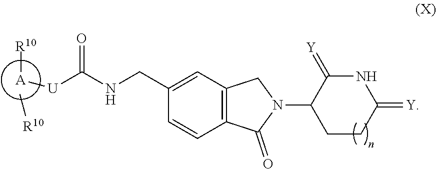

and/or pharmaceutically acceptable salts thereof, wherein:
n is 0 or 1;
A is phenyl or a C₄-C₁₀cycloalkyl ring;
U is selected from NH and CF₂;
R¹ is independently selected from hydrogen and halo;
R¹⁰ is selected from —CH₃, —C(O)R³⁰, —N(R⁴⁰)₂, —(CH₂)_{n′}OH, —(CH₂)_{n′}N(R⁴⁰)₂, —(CH₂)_{n′}Q(CH₂)_{m′}OH, —(CH₂)_{n′}Q(CH₂)_{m′}SH, and —(CH₂)_{n′}Q(CH₂)_{m′}N(R⁴⁰)₂; wherein:
   R³⁰ is hydrogen or C₁-C₆alkyl;
   each R⁴⁰ is independently hydrogen or C₁-C₆alkyl;
   Q is O, S, or NR⁴⁰;
   n′ is 1-6; and
   m′ is 2-5; and
each Y is independently S or O.

As used herein, the term “C₂-C₆alkenyl” refers to a group derived from a straight or branched chain hydrocarbon containing from two to six carbon atoms and at least one carbon-carbon double bond.

As used herein, the term “C₁-C₆alkoxy,” as used herein, refers to a C₁-C₆alkyl group attached through an oxygen atom.

As used herein, the term “C₁-C₆alkoxyC₁-C₆alkyl” refers to a C₁-C₆alkoxy group attached through a C₁-C₆alkyl group.

As used herein, the term “C₁-C₆alkynyl” refers to a group derived from a straight or branched chain hydrocarbon containing from two to six carbon atoms and at least one carbon-carbon triple bond.

As used herein, the term “C₂-C₆alkenyl” refers to a group derived from a straight or branched chain saturated hydrocarbon containing from two to six carbon atoms and at least one carbon-carbon double bond.

As used herein, the term “C₃-C₆cycloalkyl” refers to a a saturated monocyclic, hydrocarbon ring system having three to six carbon atoms and zero heteroatoms. Representative examples include, but are not limited to, cyclobutyl, cyclopentyl, and cyclohexyl.

As used herein, the term “C₄-C₁₀cycloalkyl” refers to a a saturated monocyclic, hydrocarbon ring system having four to ten carbon atoms and zero heteroatoms. Representative examples of cycloalkyl groups include, but are not limited to, cyclobutyl, cyclopentyl, and cyclohexyl. The cycloalkyl groups containing between seven and ten atoms may be monocyclic or fused, spirocyclic, or bridged bicyclic structures.

As used herein, the term “C₃-C₆cycloalkyl(C₁-C₃alkyl)” refers to a C₃-C₆cycloalkyl group attached through a C₁-C₃alkyl group.

As used herein, the term “halo” refers to F, Cl, Br, or I.

### III. NeoDegrader Conjugates

The present disclosure provides conjugates of one or more neoDegraders disclosed herein and a binding moiety. These conjugates can degrade proteins by binding to cereblon (CRBN), promoting recruitment and ubiquitination of substrate proteins mediated by CRL4^{CRBN} E3 ubiquitin ligase. These agents act as “molecular glues,” filling the binding interface as a hydrophobic patch that reprograms protein interactions between the ligase and neosubstrates.

In some aspects, the present disclosure provides a conjugate of formula (I) or formula (II),

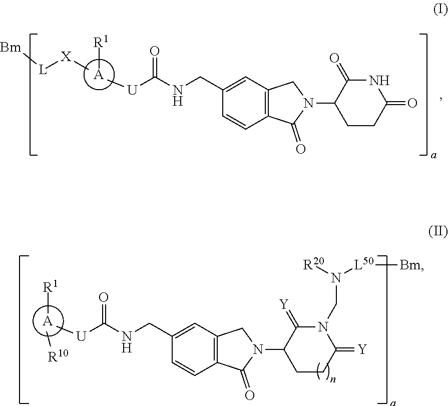

or a pharmaceutically acceptable salt thereof, wherein:
a is an integer from 1 to 10;
n is 0 or 1;
A is phenyl or a C₄-C₁₀cycloalkyl ring;
U is selected from NH, O, S, and CF₂;
R¹ is independently selected from hydrogen and halo;
R¹⁰ is selected from —CH₃, —C(O)R³⁰, —N(R⁴⁰)₂, —(CH₂)_{n′}OH, —(CH₂)_{n′}N(R⁴⁰)₂, —(CH₂)_{n′}Q(CH₂)_{m′}OH, —(CH₂)_{n′}Q(CH₂)_{m′}SH, and —(CH₂)_{n′}Q(CH₂)_{m′}N(R⁴⁰)₂, wherein:
   R³⁰ is hydrogen or C₁-C₆alkyl;
   each R⁴⁰ is independently hydrogen or C₁-C₆alkyl;
   Q is O, S, or NR⁴⁰;
   n′ is 1-6; and
   m′ is 2-5;
R²⁰ is selected from hydrogen, —(CH₂CH₂O)_{v′}—CH₃, C₂-C₆alkenyl, C₁-C₆alkyl; C₂-C₆alkynyl, benzyl, C₃-C₆cycloalkyl, and C₃-C₆cycloalkyl(C₁-C₃alkyl), wherein v′ is from 1 to 24;
X is selected from —NR²⁰⁰—, =C(CH₃)—, -Q′—(CH₂)_{n′}—, and -Q′(CH₂)_{m″}Q″(CH₂)_{n″}—; wherein:
   Q′ and Q″ are each independently O, S, or N(R²⁰⁰)ᵥ; wherein:
   v is 1 or 2; and
   each R²⁰⁰ is independently hydrogen or C₁-C₆alkyl;
   n″ is an integer from 1 to 6; and
   m″ is an integer from 2 to 6;
wherein the left side of each X group is attached to L and the right side is attached to A;
provided that when X is NH or -Q′—(CH₂)_{n′}—, R¹ is halo;
each Y is independently S or 0;
L is a cleavable linker or non-cleavable linker;
L⁵⁰ is a cleavable linker;
and
Bm is a binding moiety that is capable of specifically binding to CD56.

### III.A. Linker

The neoDegraders of the present disclosure can be linked to the binding moiety via a linker. As used herein, the term “linker” refers to any chemical moiety capable of connecting the binding moiety (Bm) to group X within the conjugates of formula (I) or to the nitrogen atom of the glutaramide ring within the conjugates of formula (II).

In certain aspects, the linkers can contain a heterobifunctional group. In the present disclosure, the term “heterobifunctional group” refers to a chemical moiety that connects the linker of which it is a part to the binding moiety. Heterobifunctional groups are characterized as having different reactive groups at either end of the chemical moiety. Attachment to “Bm,” can be accomplished through chemical or enzymatic conjugation, or a combination of both. Chemical conjugation involves the controlled reaction of accessible amino acid residues on the surface of the binding moiety with a reaction handle on the heterobifunctional group. Examples of chemical conjugation include, but are not limited to, lysine amide coupling, cysteine coupling, and coupling via a non-natural amino acid incorporated by genetic engineering, wherein non-natural amino acid residues with a desired reaction handle are installed onto “Bm.” In enzymatic conjugation, an enzyme mediates the coupling of the linker with an accessible amino residue on the binding moiety. Examples of enzymatic conjugation include, but are not limited to, transpeptidation using sortase, transpeptidation using microbial transglutaminase, and N-glycan engineering. Chemical conjugation and enzymatic conjugation may also be used sequentially. For example, enzymatic conjugation can also be used for installing unique reaction handles on “Bm” to be utilized in subsequent chemical conjugation.

In some aspects, the heterobifunctional group is selected from:

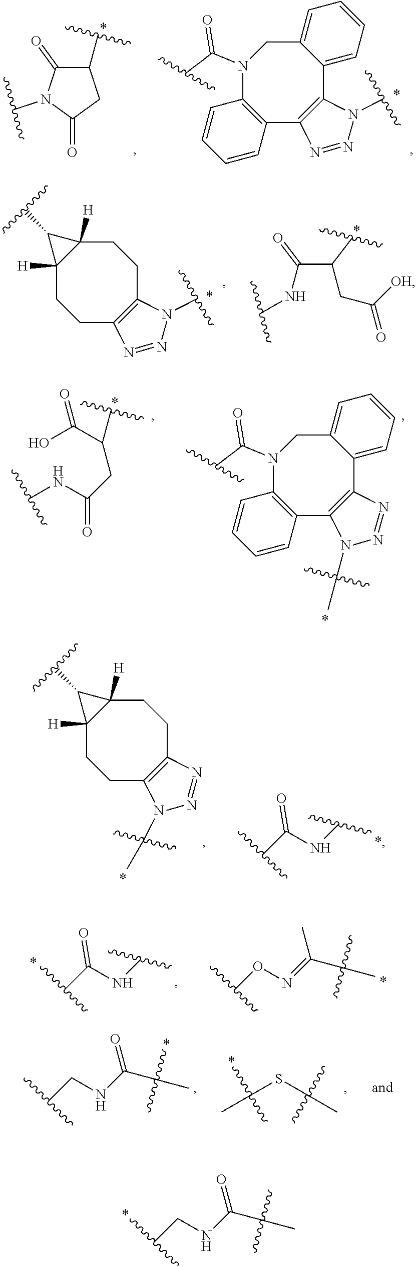

wherein:
[symbol] is the point of attachment to the remaining portion of the linker; and
[symbol] the point of attachment to Bm.

In certain aspects, linker “L” is non-cleavable. As used here, the term “non-cleavable linker” is any chemical moiety that is capable of linking the binding moiety to the neoDegrader in a stable, covalent manner and does not fall under the categories defined herein as “cleavable linkers”. Thus, non-cleavable linkers are substantially resistant to acid-induced cleavage, light-induced cleavage, bioreductive cleavage, peptidase-induced cleavage, esterase-induced cleavage, and disulfide bond cleavage. “Substantially resistant to cleavage” means that the chemical bond in the linker or adjoining the linker in at least 80%, preferably at least 85%, more preferably at least 90%, even more preferably at least 95%, and most preferably at least 99% of the antibody neoDegrader conjugate population remains non-cleavable by an acid, a photolabile-cleaving agent, a bioreductive agent, a peptidase, an esterase, or a chemical or a physiological compound that cleaves the chemical bond (for example, a disulfide bond) in a cleavable linker, for within a few hours to several days of treatment with any of the agents described above. In certain aspects the linker is not susceptible to acid-induced cleavage, photo-induced cleavage, bioreductive cleavage, enzymatic cleavage, or the like, at conditions under which the neoDegrader and/or binding moiety can remain active. NeoDegrader conjugate catabolites generated from non-cleavable linkers contain a residual amino acid from the antibody. These catabolites can exert unique and unexpected properties in the target cells to which they are delivered.

A person of ordinary skill in the art would readily distinguish non-cleavable from cleavable linkers.

Examples of non-cleavable linkers include, but are not limited to, SMCC (succinimidyl 4-(N-maleimidomethyl)cyclohexane-1-carboxylate) linkers, succinimide thioether linkers, and linkers such as:

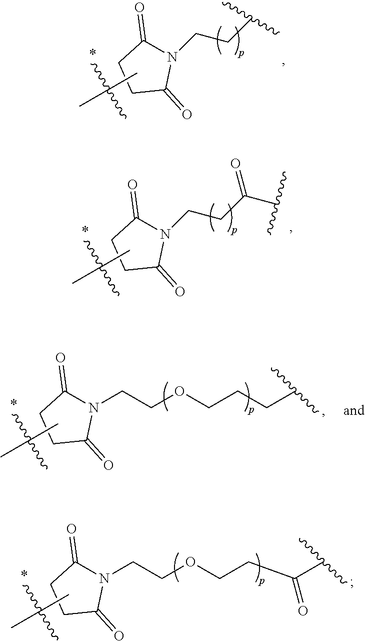

wherein:
p is an integer from 1 to 10;
[symbol] is the point of attachment to X; and
[symbol] is the point of attachment to the binding moiety.

In some aspects, L is:

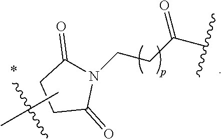

In some aspects, p is 5.

In certain aspects L and L⁵⁰ can be cleavable linkers. In some aspects, the linkers can be susceptible to acid-induced cleavage, photo-induced cleavage, bioreductive cleavage, enzymatic cleavage, or the like, at conditions under which the neoDegrader and/or binding moiety can remain active.

In some aspects, the cleavable linkers can be cleaved enzymatically. In some aspects, the cleavable linker can be cleaved by a protease, peptidase, esterase, beta-glucuronidase, glycosidase, phosphodiesterase, phosphatase, pyrophosphatase, or lipase.

In some aspects, the cleavable linkers can be cleaved by a protease. Examples of proteases include, but are not limited to, cathepsin B, VAGP tetrapeptide, and the like.

In certain aspects, the cleavable linkers contain a peptide. In some aspects, the peptide is the site of cleavage of the linker, thereby facilitating release of the drug upon exposure to intracellular proteases, such as lysosomal enzymes. Peptides can be designed and optimized for enzymatic cleavage by a particular enzyme, for example, a tumor-associated protease, cathepsin B, C and D, or a plasmin protease. Examples of peptides having two amino acids include, but are not limited to, alanine-alanine (ala-ala), valine-alanine (val-ala), valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Examples of peptides having three amino acids include, but are not limited to, glycine-valine-citrulline (gly-val-cit), aspartic acid-valine-citrulline (asp-val-cit), alanine-alanine-asparagine (ala-ala-asn), alanine-phenylalanine-lysine (ala-phe-lys), glycine-glycine-phenylalanine (gly-gly-phe), and glycine-glycine-glycine (gly-gly-gly). Examples of peptides having four amino acids include, but are not limited to, glycine-glycine-valine-citrulline (gly-gly-val-cit) and glycine-glycine-phenylalanine-glycine (gly-gly-phe-gly). Examples of peptides having five amino acids include, but are not limited to, glycine-glycine-valine-citrulline-glycine (gly-gly-val-cit-gly) and glycine-glycine-phenylalanine-glycine-glycine (gly-gly-phe-gly-gly). The amino acid combinations above can also be present in the reverse order (i.e., cit-val).

The peptides of the present disclosure can comprise L- or D-isomers of amino acid residues. The term “naturally-occurring amino acid” refers to Ala, Asp, Asx, Cit, Cys, Glu, Phe, Glx, Gly, His, Ile, Lys, Leu, Met, Asn, Pro, Gln, Arg, Ser, Thr, Val, Trp, and Tyr. “D-” designates an amino acid having the “D” (dextrorotary) configuration, as opposed to the configuration in the naturally occurring (“L-”) amino acids. The amino acids described herein can be purchased commercially (Sigma Chemical Co., Advanced Chemtech) or synthesized using methods known in the art.

In certain aspects, linkers L and L⁵⁰ are protease cleavable linkers selected from

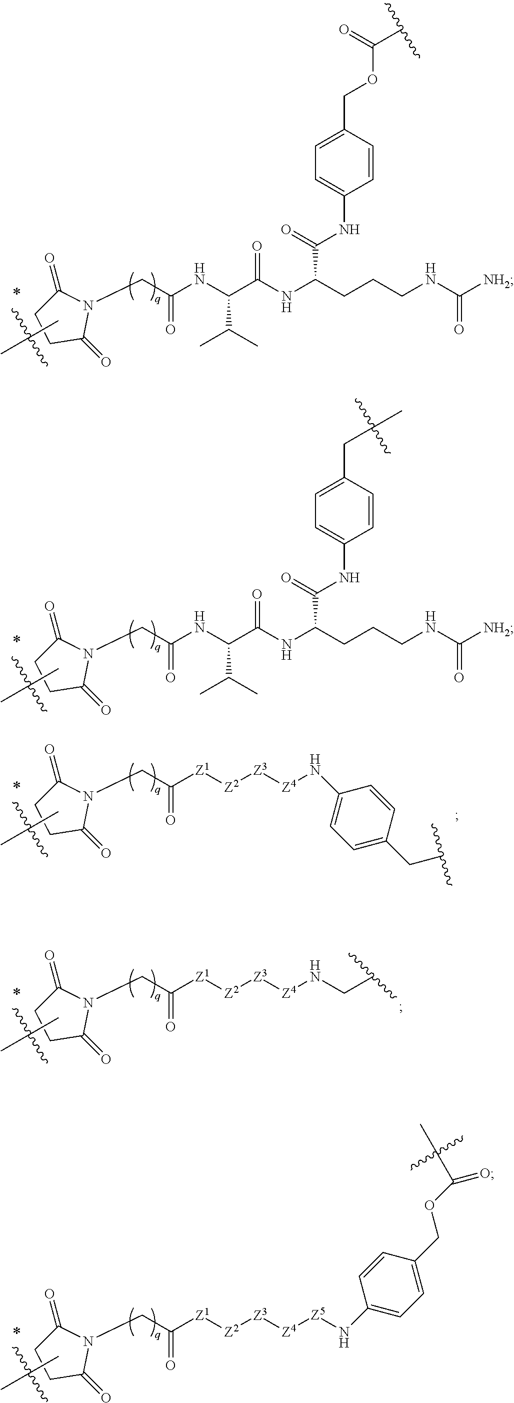

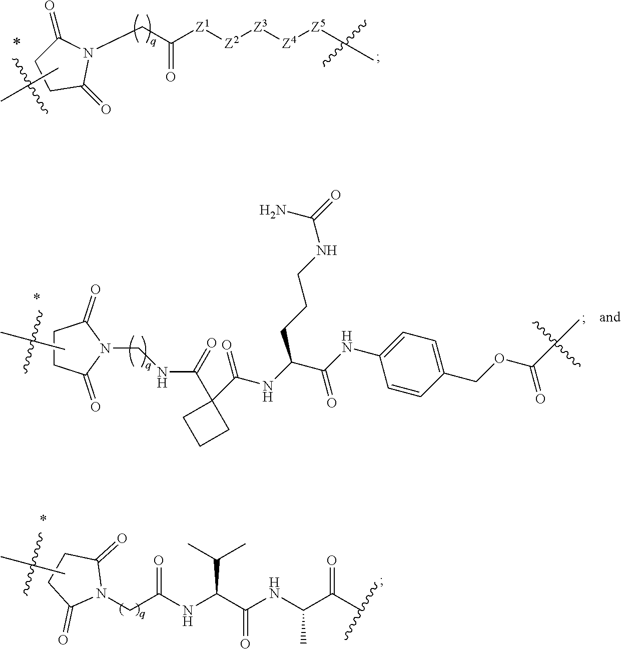

wherein:
q is an integer from 2 to 10;
Z¹, Z², Z³, Z⁴, and Z⁵ are each independently absent or a naturally-occurring amino acid residue in the L- or D-configuration, provided that at least two of Z¹, Z², Z³, Z⁴, and Z⁵ are amino acid residues;
[symbol] is the point of attachment to group X within the conjugates of formula (I) or to the nitrogen atom of the glutaramide ring within the conjugates of formula (II); and
[symbol] is the point of attachment to the binding moiety.

In certain aspects, Z¹, Z², Z³, Z⁴, and Z⁵ are independently absent or selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, Z⁴, and Z⁵ are amino acid residues.

In some aspects, Z¹ is absent or glycine; Z² is absent or selected from L-glutamine, D-glutamine, L-glutamic acid, D-glutamic acid, L-aspartic acid, D-aspartic acid, L-alanine, D-alanine, and glycine; Z³ is selected from L-valine, D-valine, L-alanine, D-alanine, L-phenylalanine, D-phenylalanine, and glycine; Z⁴ is selected from L-alanine, D-alanine, L-citrulline, D-citrulline, L-asparagine, D-asparagine, L-lysine, D-lysine, L-phenylalamine, D-phenylalanine, and glycine; and Z⁵ is absent or glycine.

In some aspects, L is

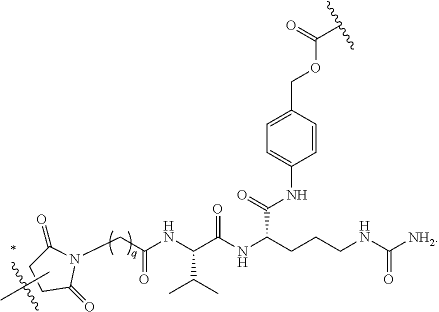

In some aspects, q is 5.

In some aspects, L⁵⁰ is

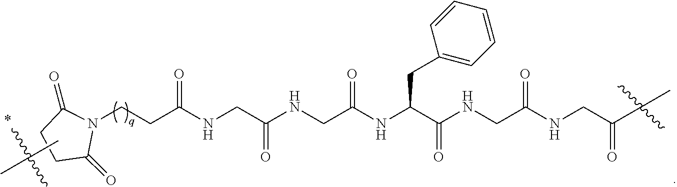

In some aspects, q is 4.

In certain aspects, L is a pyrophosphatase cleavable linker.

In some aspects, L is a pyrophosphatase cleavable linker which is:

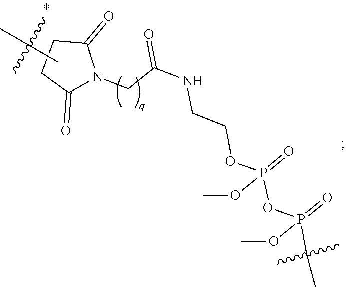

wherein:
q is an integer from 2 to 10;
[symbol] is the point of attachment to X; and
[symbol] is the point of attachment to the binding moiety.

In certain aspects, L is a beta-glucuronidase cleavable linker.

In some aspects, L is a beta-glucuronidase cleavable linker selected from:

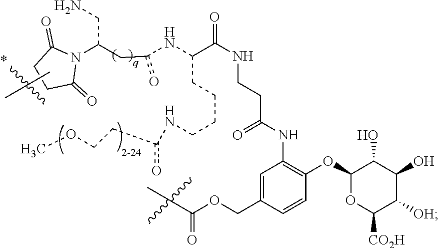

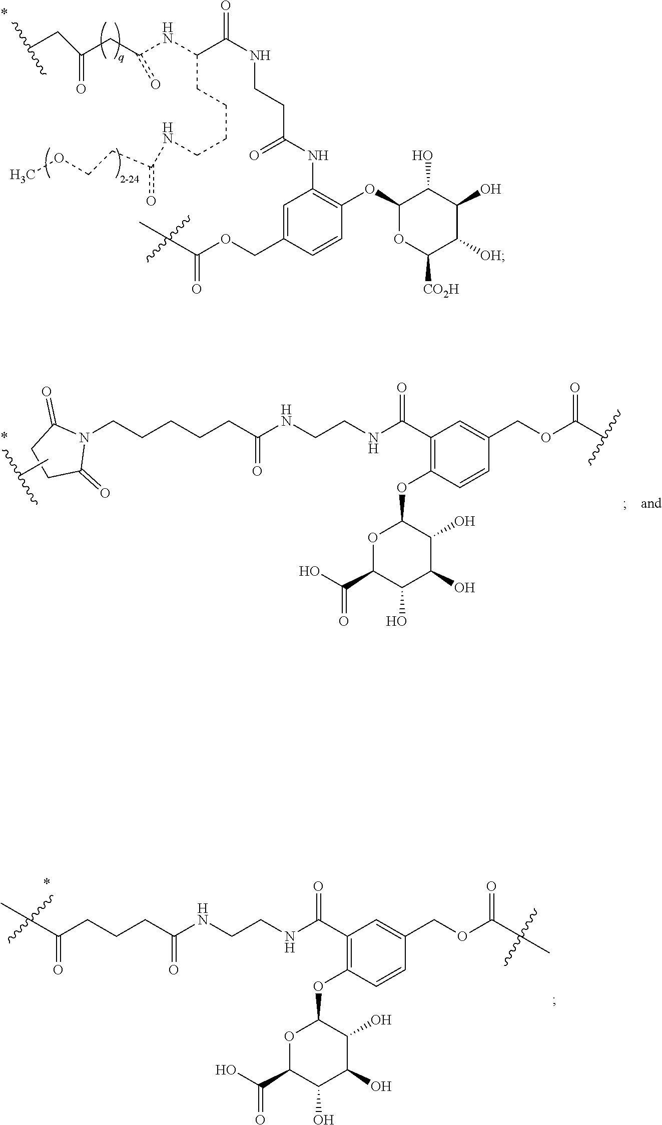

wherein:
q is an integer from 2 to 10;
[symbol] is absent or a bond;
[symbol] is the point of attachment to X; and
[symbol] is the point of attachment to the binding moiety.

In some aspects, L is

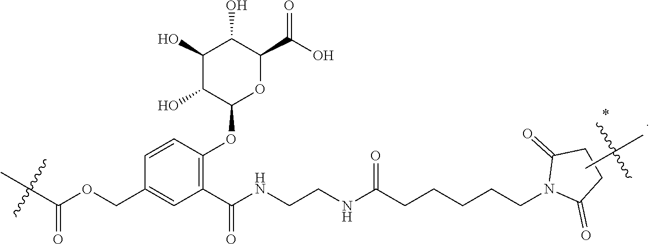

In certain aspects, L is a beta-glucuronidase cleavable linker.

In some aspects, L⁵⁰ is a beta-glucuronidase cleavable linker selected from

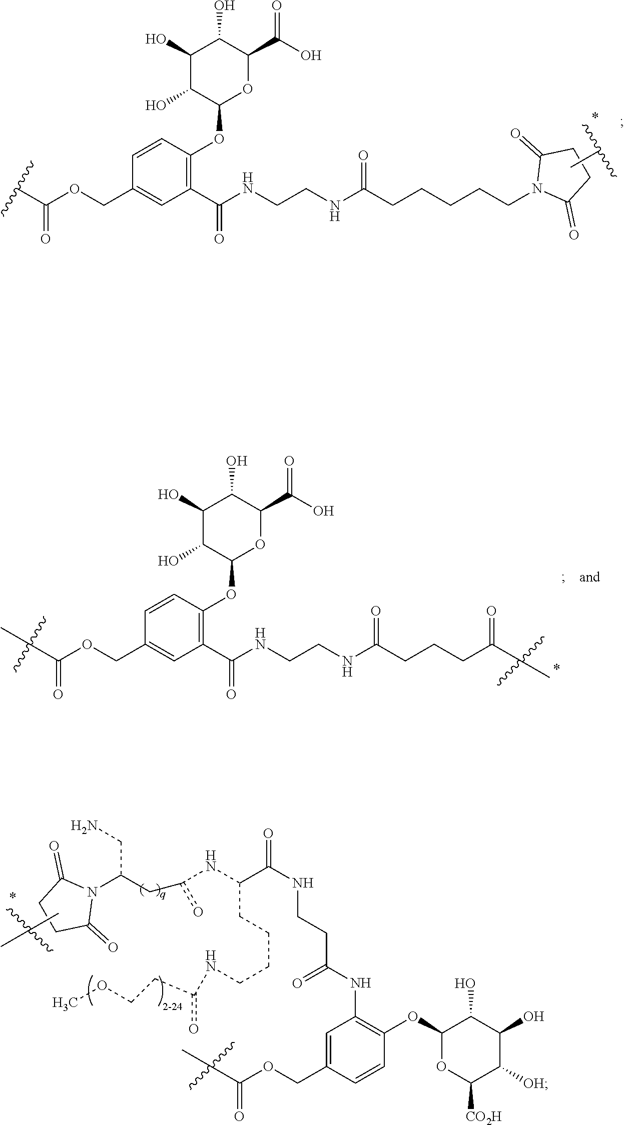

wherein:
q is from 2 to 10;
[symbol] is absent or a bond;
[symbol] is the point of attachment to the nitrogen atom of the glutaramide ring within the conjugates of formula (II); and
[symbol] is the point of attachment to the binding moiety.

In some aspects, L and L⁵⁰ are bioreducible linkers. Bioreducible linkers take advantage of the difference in reduction potential in the intracellular compartment versus plasma. Reduced glutathione presented in tumor cells' cytoplasm is up to 1000-fold higher than that present in normal cells' cytoplasm, and the tumor cells also contain enzymes which can contribute to reduction in cellular compartments. The linkers keep conjugates intact during systemic circulation, and are selectively cleaved by the high intracellular concentration of glutathione, releasing the active drugs at the tumor sites from the non-toxic prodrugs.

In some aspects, L and L⁵⁰ are bioreducible linkers selected from:

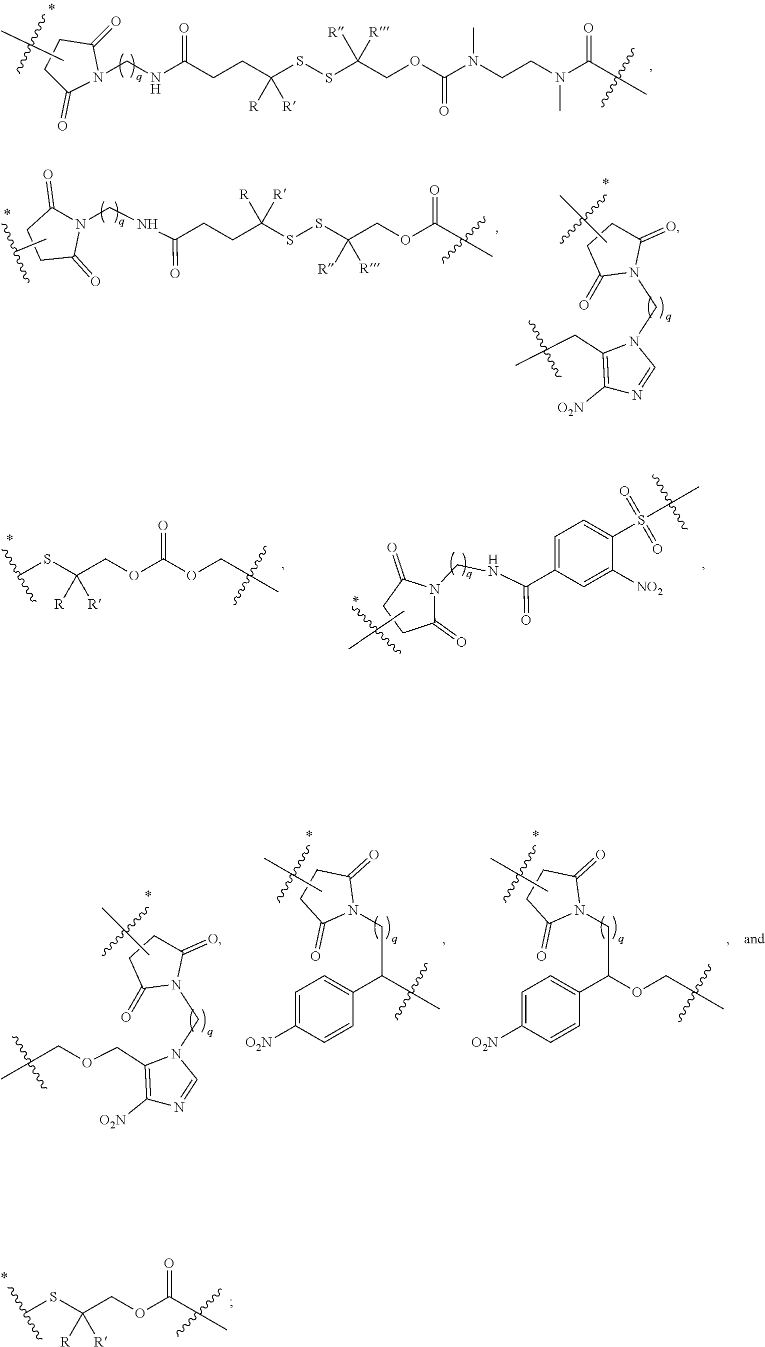

wherein:
q is an integer from 2 to 10;
R, R′, R″, and R′″ are each independently selected from hydrogen, C₁-C₆alkoxyC₁-C₆alkyl, (C₁-C₆alkyl)₂NC₁-C₆alkyl, and C₁-C₆alkyl, or, two geminal R groups, together with the carbon atom to which they are attached, can form a cyclobutyl or cyclopropyl ring;
[symbol] is the point of attachment to group X within the conjugates of formula (I) or to the nitrogen atom of the glutaramide ring within the conjugates of formula (II); and
[symbol] is the point of attachment to the binding moiety.

In some aspects, L⁵⁰ is

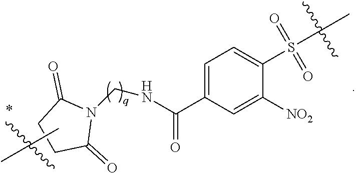

In some aspects, q is 2.

In certain aspects, L is an acid-cleavable linker. Acid-cleavable linkers are specifically designed to remain stable at the neutral pH of blood circulation, but undergo hydrolysis and release the cytotoxic drug in the acidic environment of the cellular compartments.

In some aspects, L is an acid-cleavable linker selected from

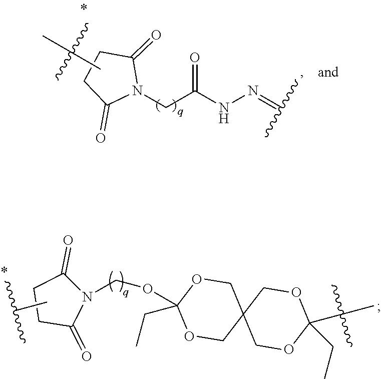

wherein:
q is an integer from 2 to 10;
[symbol] is the point of attachment to X; and
[symbol] is the point of attachment to the binding moiety.

In certain aspects, L and L⁵⁰ are click-to-release linkers, where release of the neoDegrader is chemically triggered by a tetrazine or related compound.

In some aspects, Land L⁵⁰ are click-to-release linkers selected from

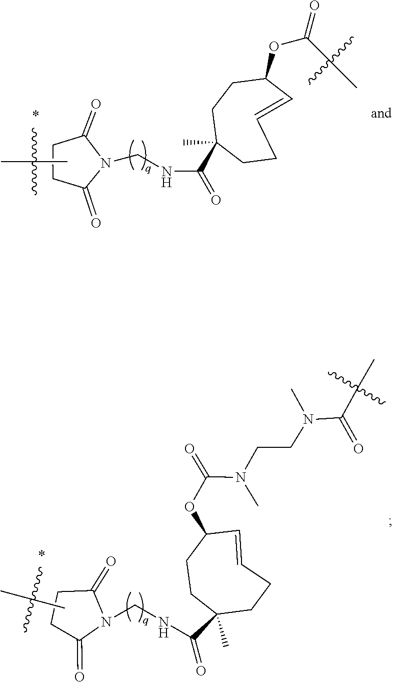

wherein:
q is an integer from 2 to 10;
[symbol] is the point of attachment to group X within the conjugates of formula (I) or to the nitrogen atom of the glutaramide ring within the conjugates of formula (II); and
[symbol] is the point of attachment to the binding moiety.

### III.B. Binding Moiety

The present disclosure provides neoDegraders conjugated to binding moieties. The term “binding moiety,” as used herein, refers to any molecule that recognizes and binds to a cell surface marker or receptor. In certain aspects, the binding moiety binds to a CD56. The binding moiety, in addition to targeting the neoDegrader to a specific cell, tissue, or location, can also have certain therapeutic effect such as antiproliferative (cytostatic and/or cytotoxic) activity against a target cell or pathway. In certain aspects the binding moiety can comprise or can be engineered to comprise at least one chemically reactive group such as a carboxylic acid, amine, thiol, or chemically reactive amino acid moiety or side chain. In some aspects, the binding moiety can comprise a targeting moiety which binds or complexes with CD56, for a given target cell population. Following specific binding or complexing with CD56, a cell expressing CD56 is permissive for uptake of the neoDegrader conjugate, which is then internalized into the cell.

In some aspects, group “Bm” can be a moiety that can specifically bind to CD56. In some aspects, group “Bm” can be a peptide or a protein that binds to CD56

In certain aspects, group “Bm” can be an antibody, antibody fragment, or an antigen-binding fragment. An antibody is a protein generated by the immune system that is capable of recognizing and binding to a specific antigen. A target antigen generally has numerous binding sites, also called epitopes, recognized by CDRs on multiple antibodies. Each antibody that specifically binds to a different epitope has a different structure. Thus, one antigen may have more than one corresponding antibody. The term “antibody” herein is used in the broadest sense and specifically covers monoclonal antibodies, single domain antibodies, polyclonal antibodies, multispecific antibodies (e.g., bispecific antibodies), and antibody fragments, so long as they exhibit the desired biological activity. Antibodies may be murine, human, humanized, chimeric, or derived from other species.

Monoclonal antibodies that can be conjugated to the neoDegrader are homogeneous populations of antibodies to a particular antigenic determinant (e.g., CD56). A monoclonal antibody (mAb) to an antigen-of-interest can be prepared by using any technique known in the art which provides for the production of antibody molecules by continuous cell lines in culture. These include, but are not limited to, the hybridoma technique, the human B cell hybridoma technique, and the EBV-hybridoma technique. Such antibodies may be of any immunoglobulin class including IgG, IgM, IgE, IgA, and IgD and any subclass thereof. The hybridoma producing the mAbs of use in this disclosure may be cultivated in vitro or in vivo.

Useful monoclonal antibodies include, but are not limited to, human monoclonal antibodies, humanized monoclonal antibodies, antibody fragments, or chimeric human-mouse (or other species) monoclonal antibodies. Human monoclonal antibodies may be made by any of numerous techniques known in the art.

The antibody can also be a bispecific antibody. Methods for making bispecific antibodies are known in the art. Traditional production of full-length bispecific antibodies is based on the coexpression of two immunoglobulin heavy chain-light chain pairs, where the two chains have different specificities. Because of the random assortment of immunoglobulin heavy and light chains, these hybridomas (quadromas) produce a potential mixture of 10 different antibody molecules, of which only one has the correct bispecific structure. Purification of the correct molecule, which is usually performed using affinity chromatography steps, is rather cumbersome, and the product yields are low.

According to a different approach, antibody variable domains with the desired binding specificities (antibody-antigen combining sites) are fused to immunoglobulin constant domain sequences. The fusion may be with an immunoglobulin heavy chain constant domain, comprising at least part of the hinge, C_{H2}, and C_{H3} regions. The first heavy-chain constant region (C_{H1}) may contain the site necessary for light chain binding, present in at least one of the fusions. Nucleic acids with sequences encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in aspects when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

Bispecific antibodies may have a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. Using such techniques, bispecific antibodies can be prepared for conjugation to the neoDegraders in the treatment or prevention of disease as defined herein.

Hybrid or bifunctional antibodies can be derived either biologically, i.e., by cell fusion techniques, or chemically, especially with cross-linking agents or disulfide-bridge forming reagents, and may comprise whole antibodies or fragments thereof.

The antibody can be a functionally active fragment, derivative or analog of an antibody that immunospecifically binds to cancer cell antigens, viral antigens, or microbial antigens or other antibodies bound to tumor cells or matrix. In this regard, “functionally active” means that the fragment, derivative or analog is able to elicit anti-anti-idiotype antibodies that recognize the same antigen that the antibody from which the fragment, derivative or analog is derived recognized. Specifically, in an exemplary aspect the antigenicity of the idiotype of the immunoglobulin molecule can be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art.

Other useful antibodies include fragments of antibodies such as, but not limited to, F(ab′)2 fragments, which contain the variable region, the light chain constant region and the CH1 domain of the heavy chain can be produced by pepsin digestion of the antibody molecule, and Fab fragments, which can be generated by reducing the disulfide bridges of the F(ab′)2 fragments. Other useful antibodies are heavy chain and light chain dimers of antibodies, or any minimal fragment thereof such as Fvs or single chain antibodies (SCAs), or any other molecule with the same specificity as the antibody.

Additionally, recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as those having a variable region derived from a murine monoclonal and human immunoglobulin constant regions. Humanized antibodies are antibody molecules from non-human species having one or more complementarity determining regions (CDRs) from the non-human species and a framework region from a human immunoglobulin molecule. Such chimeric and humanized monoclonal antibodies can be produced by recombinant DNA techniques known in the art.

Completely human antibodies can be produced using transgenic mice that are incapable of expressing endogenous immunoglobulin heavy and light chains genes, but which can express human heavy and light chain genes. The transgenic mice are immunized in the normal fashion with a selected antigen, e.g., all or a portion of a polypeptide of the disclosure. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). Other human antibodies can be obtained commercially from, for example, Abgenix, Inc. (Freemont, Calif.) and Genpharm (San Jose, Calif.).

Completely human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope. Human antibodies can also be produced using various techniques known in the art, including phage display libraries.

The antibody can be a fusion protein of an antibody, or a functionally active fragment thereof, for example in which the antibody is fused via a covalent bond (e.g., a peptide bond), at either the N-terminus or the C-terminus to an amino acid sequence of another protein (or portion thereof, such as at least 10, 20 or 50 amino acid portion of the protein) that is not the antibody. The antibody or fragment thereof may be covalently linked to the other protein at the N-terminus of the constant domain.

Antibodies include analogs and derivatives that are either modified, i.e., by the covalent attachment of any type of molecule as long as such covalent attachment permits the antibody to retain its antigen binding immunospecificity. For example, but not by way of limitation, the derivatives and analogs of the antibodies include those that have been further modified, e.g., by glycosylation, acetylation, pegylation, phosphorylation, amidation, derivatization by known protecting/blocking groups, proteolytic cleavage, linkage to a cellular antibody unit or other protein, etc. Any of numerous chemical modifications can be carried out by known techniques, including, but not limited to specific chemical cleavage, acetylation, formylation, metabolic synthesis in the presence of tunicamycin, etc. Additionally, the analog or derivative can contain one or more unnatural amino acids.

The antibodies in neoDegrader conjugates can include antibodies having modifications (e.g., substitutions, deletions or additions) in amino acid residues that interact with Fc receptors. In particular, antibodies include antibodies having modifications in amino acid residues identified as involved in the interaction between the anti-Fc domain and the FcRn receptor. Antibodies immunospecific for a cancer cell antigen can be obtained commercially or produced by any method known to one of skill in the art such as, e.g., chemical synthesis or recombinant expression techniques. The nucleotide sequence encoding antibodies immunospecific for a cancer cell antigen can be obtained, e.g., from the GenBank database or a database like it, the literature publications, or by routine cloning and sequencing.

In certain aspects, the antibody of the neoDegrader conjugates can be a monoclonal antibody, e.g., a murine monoclonal antibody, a chimeric antibody, or a humanized antibody. In some aspects, the antibody can be an antibody fragment, e.g., a Fab fragment.

Known anti-CD56 antibodies can be conjugated to the neoDegraders described herein. Antibodies immunospecific for a CD56-expressing cancer cell can be obtained commercially or produced by any method known to one of skill in the art such as, e.g., recombinant expression techniques. The nucleotide sequence encoding antibodies immunospecific for a cancer cell antigen can be obtained, e.g., from the GenBank database or a database like it, the literature publications, or by routine cloning and sequencing. An example of an anti-CD56 antibody is lorvotuzumab.

In some aspects, a binding moiety is an antibody or antigen binding fragment thereof that comprises the 6 CDRs of an antibody in Table A (i.e., the 3 CDRs of the variable heavy chain or heavy chain and the 3 CDRs of the variable light chain or light chain of the same antibody).

The term “Kabat numbering” and like terms are recognized in the art and refer to a system of numbering amino acid residues in the heavy and light chain variable regions of an antibody or an antigen-binding fragment thereof. In some aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In some aspects, a binding moiety is an antibody or antigen binding fragment thereof that comprises the 6 Kabat-defined CDRs of an antibody in Table A (i.e., the 3 Kabat-defined CDRs of the variable heavy chain or heavy chain and the 3 Kabat-defined CDRs of the variable light chain or light chain of the same antibody).

The CDRs of an antibody or antigen-binding fragment thereof can be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In some aspects, a binding moiety is an antibody or antigen binding fragment thereof that comprises the 6 Chothia-defined CDRs of an antibody in Table A (i.e., the 3 Chothia-defined CDRs of the variable heavy chain or heavy chain and the 3 Chothia-defined CDRs of the variable light chain or light chain of the same antibody). In some aspects, a binding moiety is an antibody or antigen binding fragment thereof that comprises one or more CDRs, in which the Chothia and Kabat CDRs have the same amino acid sequence. In some aspects, a binding moiety is an antibody or antigen binding fragment thereof that comprises a combination of Kabat CDRs and Chothia CDRs of an antibody in Table A.

In some aspects, the CDRs of an antibody or antigen-binding fragment thereof can be determined according to the IMGT numbering system as described in Lefranc M-P, (1999) The Immunologist 7: 132-136 and Lefranc M-P et al., (1999) Nucleic Acids Res 27: 209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97. In some aspects, a binding moiety is an antibody or antigen binding fragment thereof that comprises the 6 IMGT-defined CDRs of an antibody in Table A (i.e., the 3 IMGT-defined CDRs of the variable heavy chain or heavy chain and the 3 IMGT-defined CDRs of the variable light chain or light chain of the same antibody), for example, as described in Lefranc M-P (1999) supra and Lefranc M-P et al., (1999) supra).

In some aspects, the CDRs of an antibody or antigen-binding fragment thereof can be determined according to MacCallum R M et al., (1996) J Mol Biol 262: 732-745. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In some aspects, a binding moiety is an antibody or antigen binding fragment thereof that comprises the 6 MacCallum-defined CDRs of an antibody in Table A (i.e., the 3 MacCallum-defined CDRs of the variable heavy chain or heavy chain and the 3 MacCallum-defined CDRs of the variable light chain or light chain of the same antibody), for example as determined by the method in MacCallum R M et al.

In some aspects, the CDRs of an antibody or antigen-binding fragment thereof can be determined according to the AbM numbering scheme, which refers AbM hypervariable regions which represent a compromise between the Kabat CDRs and Chothia structural loops, and are used by Oxford Molecular's AbM antibody modeling software (Oxford Molecular Group, Inc.). In some aspects, a binding moiety is an antibody or antigen binding fragment thereof that comprises the 6 AbM-defined CDRs of an antibody in Table A (i.e., the 3 AbM-defined CDRs of the variable heavy chain or heavy chain and the 3 AbM-defined CDRs of the variable light chain or light chain of the same antibody) as determined by the AbM numbering scheme.

In some aspects, a binding moiety is an antibody or antigen binding fragment thereof that binds to CD56. In some aspects, an antibody or antigen binding fragment thereof binds to CD56 and comprises the 6 CDRs of an anti-CD56 antibody provided in Table A (e.g., 6 CDRs comprising the amino acid sequences of SEQ ID NOs:2-7). In some aspects, an antibody or antigen binding fragment thereof binds to CD56 and comprises a VH of an anti-CD56 antibody provided in Table A (e.g., a VH comprising the amino acid sequence of SEQ ID NO:8). In some aspects, an antibody or antigen binding fragment thereof binds to CD56 and comprises a VL of an anti-CD56 antibody provided in Table A (e.g., a VH comprising the amino acid sequence of SEQ ID NO:9). In some aspects, an antibody or antigen binding fragment thereof binds to CD56 and comprises a VH and a VL of an anti-CD56 antibody provided in Table A (e.g., a VH comprising the amino acid sequence of SEQ ID NO:8 and a VL comprising the amino acid sequence of SEQ ID NO:9). In some aspects, an antibody or antigen binding fragment thereof binds to CD56 and comprises a heavy chain comprising the amino acid sequence of SEQ TD NO:10 and a light chain comprising the amino acid sequence of SEQ ID NO: 11. In some aspects, an antibody or antigen binding fragment thereof binds to CD56 and comprises a heavy chain comprising the amino acid sequence of SEQ ID NO: 12 and a light chain comprising the amino acid sequence of SEQ ID NO: 11.

TABLE A
Exemplary Antibody or Antigen binding
fragment Thereof Sequences

| | |
|---|---|
| VH- | SFGMH (SEQ ID NO: 2) |
| CDR1 | |
| VH- | YISSGSFTIYYADSVKG (SEQ ID NO: 3) |
| CDR2 | |
| VH- | MRKGYAMDY (SEQ ID NO: 4) |
| CDR3 | |
| VL- | RSSQIIIHSDGNTYLE (SEQ ID NO: 5) |
| CDR1 | |
| VL- | KVSNRFS (SEQ ID NO: 6) |
| CDR2 | |
| VL- | FQGSHVPHT (SEQ ID NO: 7) |
| CDR3 | |
| VH | QVQLVESGGGVVQPGRSLRLSCAASGFTFSSFGMHWV |
| | RQAPGKGLEWVAYISSGSFTIYYADSVKGRFTISRDN |
| | SKNTLYLQMNSLRAEDTAVYYCARMRKGYAMDYWGQG |
| | TLVTVSS (SEQ ID NO: 8) |
| VL | DVVMTQSPLSLPVTLGQPASISCRSSQIIIHSDGNTY |
| | LEWFQQRPGQSPRRLIYKVSNRFSGVPDRFSGSGSGT |
| | DFTLKISRVEAEDVGVYYCFQGSHVPHTFGQGTKVE |
| | IK (SEQ ID NO: 9) |
| Heavy | QVQLVESGGGVVQPGRSLRLSCAASGFTFSSFGMHWV |
| chain | RQAPGKGLEWVAYISSGSFTIYYADSVKGRFTISRDN |
| of | SKNTLYLQMNSLRAEDTAVYYCARMRKGYAMDYWGQG |
| CD56- | TLVTVSSASTKGPSVFPLAPSSKSTSGGTAALGCLVK |
| A | DYFPEPVTVSWNSGALTSGVHTFPAVLQSSGLYSLSS |
| | VVTVPSSSLGTQTYICNVNHKPSNTKVDKKVEPKSCD |
| | KTHTCPPCPAPELLGGPSVFLFPPKPKDTLMISRTPE |
| | VTCVVVDVSHEDPEVKFNWYVDGVEVHNAKTKPREEQ |
| | YNSTYRVVSVLTVLHQDWLNGKEYKCKVSNKALPAPI |
| | EKTISKAKGQPREPQVYTLPPSRDELTKNQVSLTCLV |
| | KGFYPSDIAVEWESNGQPENNYKTTPPVLDSDGSFFL |
| | YSKLTVDKSRWQQGNVFSCSVMHEALHNHYTQKSLSL |
| | SPG (SEQ ID NO: 10) |
| Light | DVVMTQSPLSLPVTLGQPASISCRSSQIIIHSDGNTY |
| chain | LEWFQQRPGQSPRRLIYKVSNRFSGVPDRFSGSGSGT |
| of | DFTLKISRVEAEDVGVYYCFQGSHVPHTFGQGTKVEI |
| CD56- | KRTVAAPSVFIFPPSDEQLKSGTASVVCLLNNFYPRE |
| A | AKVQWKVDNALQSGNSQESVTEQDSKDSTYSLSSTLT |
| | LSKADYEKHKVYACEVTHQGLSSPVTKSFNRGEC |
| | (SEQ ID NO: 11) |
| Heavy | QVQLVESGGGVVQPGRSLRLSCAASGFTFSSFGMHWV |
| chain | RQAPGKGLEWVAYISSGSFTIYYADSVKGRFTISRDN |
| of | SKNTLYLQMNSLRAEDTAVYYCARMRKGYAMDYWGQG |
| CD56- | TLVTVSSASTKGPSVFPLAPSSKSTSGGTAALGCLVK |
| B | DYFPEPVTVSWNSGALTSGVHTFPAVLQSSGLYSLSS |
| | VVTVPSSSLGTQTYICNVNHKPSNTKVDKKVEPKSCD |
| | KTHTCPPCPAPEAAGGPSVFLFPPKPKDTLMISRTPE |
| | VTCVVVDVSHEDPEVKFNWYVDGVEVHNAKTKPREEQ |
| | YASTYRVVSVLTVLHQDWLNGKEYKCKVSNKALPAPI |
| | EKTISKAKGQPREPQVYTLPPSRDELTKNQVSLTCLV |
| | KGFYPSDIAVEWESNGQPENNYKTTPPVLDSDGSFFL |
| | YSKLTVDKSRWQQGNVFSCSVMHEALHNHYTQKSLSL |
| | SPG |
| | (SEQ ID NO: 12) |
| Light | DVVMTQSPLSLPVTLGQPASISCRSSQIIIHSDGNTY |
| chain | LEWFQQRPGQSPRRLIYKVSNRFSGVPDRFSGSGSGT |
| of | DFTLKISRVEAEDVGVYYCFQGSHVPHTFGQGTKVEI |
| CD56- | KRTVAAPSVFIFPPSDEQLKSGTASVVCLLNNFYPRE |
| B | AKVQWKVDNALQSGNSQESVTEQDSKDSTYSLSSTLT |
| | LSKADYEKHKVYACEVTHQGLSSPVTKSFNRGEC |
| | (SEQ ID NO: 11) |

In some aspects, an antibody or antigen binding fragment thereof comprises a constant region. A linker can be attached to an amino acid in the constant region. In some aspects, an antibody or antigen binding fragment thereof comprises a CH1 domain. A linker can be attached to an amino acid in a CH1 domain. In some aspects, an antibody or antigen binding fragment thereof comprises a CH2 domain. A linker can be attached to an amino acid in a CH2 domain. In some aspects, an antibody or antigen binding fragment thereof comprises a CH3 domain. A linker can be attached to an amino acid in a CH3 domain. In some aspects, an antibody or antigen binding fragment thereof comprises a CL domain. A linker can be attached to an amino acid in a CL domain.

In some aspects, a constant region, a CH1 domain, a CH2 domain, a CH3 domain, or a CL domain is an engineered constant region, CH1 domain, CH2 domain, CH3 domain or a CL domain.

In some aspects, an antibody or antigen binding fragment thereof comprises a heavy chain constant region, e.g., a human heavy chain constant region. A linker can be attached to an amino acid in a heavy chain constant region, e.g., a human heavy chain constant region. In some aspects, an antibody or antigen binding fragment thereof comprises an IgG heavy chain constant region, e.g., a human IgG heavy chain constant region. A linker can be attached to an amino acid in an IgG heavy chain constant region, e.g., a human IgG heavy chain constant region. In some aspects, an antibody or antigen binding fragment thereof comprises an IgG1 heavy chain constant region, e.g., a human IgG1 heavy chain constant region. A linker can be attached to an amino acid in an IgG1 heavy chain constant region, e.g., a human IgG1 heavy chain constant region. In some aspects, an antibody or antigen binding fragment thereof comprises an IgG4 heavy chain constant region. A linker can be attached to an amino acid in an IgG4 heavy chain constant region, e.g., a human IgG4 heavy chain constant region.

In some aspects, an antibody or antigen binding fragment thereof comprises a light chain constant region, e.g., a human light chain constant region. A linker can be attached to an amino acid in a light chain constant region, e.g., a human light chain constant region. In some aspects, an antibody or antigen binding fragment thereof comprises a kappa light chain constant region, e.g., a human kappa light chain constant region. A linker can be attached to an amino acid in a kappa light chain constant region, e.g., a human kappa light chain constant region. In some aspects, an antibody or antigen binding fragment thereof comprises a gamma light chain constant region, e.g., a human gamma light chain constant region. A linker can be attached to an amino acid in a gamma light chain constant region, e.g., a human gamma light chain constant region.

In some aspects, an antibody or antigen binding fragment thereof comprises an engineered cysteine at heavy chain position S239 according to EU numbering. A linker can be attached to S239C. In some aspects, an antibody or antigen binding fragment thereof comprises an engineered cysteine at heavy chain position K334 according to EU numbering. A linker can be attached to K334C.

In some aspects, an antibody or antigen-binding fragment there comprises an Fc domain with reduced effector function as compared to a wild-type Fc domain. Such antibodies or antigen-binding fragments thereof an have reduced toxicity. In some aspects, an antibody or antigen binding fragment thereof comprises an N297A mutation according to EU numbering. In some aspects, an antibody or antigen binding fragment thereof comprises a LALA (L234A and L235A) mutation according to EU numbering. In some aspects, an antibody or antigen binding fragment thereof comprises an N297A mutation according to EU numbering and a LALA (L234A and L235A) mutation according to EU numbering.

Accordingly, an antibody or antigen binding fragment thereof can comprise a heavy chain constant region of SEQ ID NO:13, SEQ ID NO:14, or SEQ ID NO:15.

IgG1 Heavy Chain Constant Region
(SEQ ID NO: 13)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPG
IgG1 Heavy Chain Constant Region S239C
(SEQ ID NO: 14)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPCVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPG
IgG1 Heavy Chain Constant Region K334C
(SEQ ID NO: 15)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIECTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPG

An antibody or antigen binding fragment thereof can comprise a heavy chain constant region of SEQ ID NO:16.

IgG4 Heavy Chain Constant Region S228P
(SEQ ID NO: 16)
ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPPCPAPEFLGGPSVFLFPPKPKDTLMISR
TPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVMHEALHNH
YTQKSLSLSLGK

An antibody or antigen binding fragment thereof can comprise a heavy chain constant region of SE ID NO:17.

IgG1 N297A Constant regions
(CH1-Hinge-CH2-CH3)
(SEQ ID NO: 17)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYA
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPG

An antibody or antigen binding fragment thereof can comprise a heavy chain constant region of SEQ ID NO:18.

IgG1 L234A, L235A Constant regions
(CH1-Hinge-CH2-CH3)
(SEQ ID NO: 18)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPEAAGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPG

An antibody or antigen binding fragment thereof can comprise a heavy chain constant region of SEQ ID NO:19.

IgG1 L234A, L235A, N297A Constant regions
(CH1-Hinge-CH2-CH3)
(SEQ ID NO: 19)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPEAAGGPSVFLFPPKPKDTLM
ISRTPEVTCVVVDVSHEDPEVKFNWYVDGVEVHNAKTKPREEQYA
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPG

In some aspects, a linker can be attached to heavy chain Q295 of an antibody or antigen binding fragment thereof according to EU numbering.

An antibody “which binds” a molecular target or an antigen of interest is one capable of binding that antigen with sufficient affinity such that the antibody is useful in targeting a cell expressing the antigen.

In the present disclosure, group “Bm” can be conjugated to more than one neoDegrader. In some aspects, “Bm” can be conjugated to from 1 to 10 neoDegraders. In some aspects, “Bm” can be conjugated to from 1 to 9 neoDegraders. In some aspects, “Bm” can be conjugated to from 1 to 8 neoDegraders. In some aspects, “Bm” can be conjugated to 1, 2, 3, 4, 5, 6, 7, 8, 9, or 10 neoDegraders. In some aspects, “Bm” can be conjugated to 7 or 8 neoDegraders. In some aspects, “Bm” is conjugated to 4 neoDegraders. In some aspects, “Bm” is conjugated to 5 neoDegraders. In some aspects, “Bm” is conjugated to 6 neoDegraders. In some aspects, “Bm” is conjugated to 7 neoDegraders. In some aspects, “Bm” is conjugated to 8 neoDegraders. In some aspects, “Bm” is conjugated to 9 neoDegraders.

### IV. Compositions and Methods of Using

The conjugates described herein can be in the form of pharmaceutically or pharmaceutically acceptable salts. In some aspects, such salts are derived from inorganic or organic acids or bases.

Examples of suitable acid addition salts include acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Examples of suitable base addition salts include ammonium salts; alkali metal salts, such as sodium and potassium salts; alkaline earth metal salts, such as calcium and magnesium salts; salts with organic bases, such as dicyclohexylamine salts, N-methyl-D-glucamine; and salts with amino acids such as arginine, lysine, and the like.

For example, Berge lists the following FDA-approved commercially marketed salts: anions acetate, besylate (benzenesulfonate), benzoate, bicarbonate, bitartrate, bromide, calcium edetate (ethylenediaminetetraacetate), camsylate (camphorsulfonate), carbonate, chloride, citrate, dihydrochloride, edetate (ethylenediaminetetraacetate), edisylate (1,2-ethanedisulfonate), estolate (lauryl sulfate), esylate (ethanesulfonate), fumarate, gluceptate (glucoheptonate), gluconate, glutamate, glycollylarsanilate (glycollamidophenylarsonate), hexylresorcinate, hydrabamine (N,N′-di(dehydroabietyl)ethylenediamine), hydrobromide, hydrochloride, hydroxynaphthoate, iodide, isethionate (2-hydroxyethanesulfonate), lactate, lactobionate, malate, maleate, mandelate, mesylate (methanesulfonate), methylbromide, methylnitrate, methylsulfate, mucate, napsylate (2-naphthalenesulfonate), nitrate, pamoate (embonate), pantothenate, phosphate/diphosphate, polygalacturonate, salicylate, stearate, subacetate, succinate, sulfate, tannate, tartrate, teoclate (8-chlorotheophyllinate) and triethiodide; organic cations benzathine (N,N′-dibenzylethylenediamine), chloroprocaine, choline, diethanolamine, ethylenediamine, meglumine (N-methylglucamine) and procaine; and metallic cations aluminum, calcium, lithium, magnesium, potassium, sodium and zinc.

Berge additionally lists the following non-FDA-approved commercially marketed (outside the United States) salts: anions adipate, alginate, aminosalicylate, anhydromethylenecitrate, arecoline, aspartate, bisulfate, butylbromide, camphorate, digluconate, dihydrobromide, disuccinate, glycerophosphate, hemisulfate, hydrofluoride, hydroiodide, methylenebis(salicylate), napadisylate (1,5-naphthalenedisulfonate), oxalate, pectinate, persulfate, phenylethylbarbiturate, picrate, propionate, thiocyanate, tosylate and undecanoate; organic cations benethamine (N-benzylphenethylamine), clemizole (1-p-chlorobenzyl-2-pyrrolidine-1′-ylmethylbenzimidazole), diethylamine, piperazine and tromethamine (tris(hydroxymethyl)aminomethane); and metallic cations barium and bismuth.

Pharmaceutical compositions comprising the neoDegrader conjugates described herein may also comprise suitable carriers, excipients, and auxiliaries that may differ depending on the mode of administration.

In some aspects, the pharmaceutical compositions can be formulated as a suitable parenteral dosage form. Said formulations can be prepared by various methods known in the art. The pharmaceutical compositions can be administered directly into the bloodstream, into muscle, or directly into an organ. Suitable means for parenteral administration include intravenous, intraarterial, intraperitoneal, intrathecal, intraventricular, intraurethral, intrasternal, intracranial, intramuscular, and subcutaneous. Suitable devices for parenteral administration include needle injectors, needle-free injectors, and infusion techniques.

Parenteral compositions are typically aqueous solutions which may contain excipients such as salts, carbohydrates and buffering agents. However, the composition may also be formulated a sterile non-aqueous solution or as a dried form to be used in conjunction with a suitable vehicle such as sterile pyrogen-free water.

The preparation of parenteral compositions under sterile conditions, for example, by lyophilization, can be readily accomplished using standard techniques known well to those of skill in the art.

Compositions for parenteral administration can be formulated to be immediate and/or modified release. Modified release formulations include delayed-, sustained-, pulsed-, controlled-, targeted, and programmed release. Thus, the compositions can be formulated as a solid, semi-solid, or thixotropic liquid for administration as an implanted depot providing modified release of the active agent.

The parenteral formulations can be admixed with other suitable pharmaceutically acceptable excipients used in parenteral dosage forms such as, but not limited to, preservatives.

In another aspect, the pharmaceutical compositions can be formulated as suitable oral dosage forms such as tablets, capsules, powders, pellets, suspensions, solutions, emulsions, and the like. Other suitable carriers can be present such as disintegrants, diluents, chelating agents, binders, glidants, lubricants, fillers, bulking agents, anti-adherants, and the like.

Oral dosage formulations may also contain other suitable pharmaceutical excipients such as sweeteners, vehicle/wetting agents, coloring agents, flavoring agents, preservatives, viscosity enhancing/thickening agents, and the like.

The neoDegrader conjugates described herein can be used to treat various cancers. Certain conjugates of the present disclosure can be superior in terms of efficacy expression, pharmacokinetics (e.g., absorption, distribution, metabolism, excretion), solubility (e.g., water solubility), interaction with other medicaments (e.g., drug-metabolizing enzyme inhibitory action), safety (e.g., acute toxicity, chronic toxicity, genetic toxicity, reproductive toxicity, cardiotoxicity, carcinogenicity, central toxicity) and/or stability (e.g., chemical stability, stability to an enzyme), and can be useful as a medicament.

The neoDegrader conjugates of the present disclosure can be used as medicaments such as an agents for the prophylaxis or treatment of diseases, for example, cancers e.g., CD56-positive cancers. In some aspects, the cancer is a neuroendocrine cancer (e.g., neuroblastoma or neuroendocrine prostate cancer), a lung cancer (e.g., small cell lung cancer (SCLC)), or a sarcoma.

Furthermore, neoDegrader conjugates of the present disclosure or can be used concurrently with a non-drug therapy. To be precise, the conjugates can be combined with a non-drug therapy such as (1) surgery, (2) hypertensive chemotherapy using angiotensin II etc., (3) gene therapy, (4) thermotherapy, (5) cryotherapy, (6) laser cauterization and (7) radiotherapy.

For example, by using a neoDegrader conjugate of the present disclosure before or after the above-mentioned surgery and the like, effects such as prevention of emergence of resistance, prolongation of Disease-Free Survival, suppression of cancer metastasis or recurrence, prolongation of life and the like may be afforded.

In addition, it is possible to combine a treatment with neoDegrader conjugates of the present disclosure with a supportive therapy: (i) administration of antibiotic (e.g., ®-lactam type such as pansporin and the like, macrolide type such as clarithromycin and the like) for the complication with various infectious diseases, (ii) administration of high-calorie transfusion, amino acid preparation or general vitamin preparation for the improvement of malnutrition, (iii) administration of morphine for pain mitigation, (iv) administration of a pharmaceutical agent for ameliorating side effects such as nausea, vomiting, anorexia, diarrhea, leucopenia, thrombocytopenia, decreased hemoglobin concentration, hair loss, hepatopathy, renopathy, DIC, fever and the like and (v) administration of a pharmaceutical agent for suppressing multiple drug resistance of cancer and the like.

In some aspects, the conjugates of the disclosure can be used in combination with a standard of care therapy, e.g., one or more therapeutic agents (e.g., anti-cancer agents and/or immunomodulating agents). Accordingly, in certain aspects, a method of treating a tumor disclosed herein comprises administering the conjugates of the disclosure in combination with one or more additional therapeutic agents. In some aspects, the conjugates of the disclosure can be used in combination with one or more anti-cancer agents, such that multiple elements of the immune pathway can be targeted. In some aspects, an anti-cancer agent comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof. A comprehensive and non-limiting list of combination treatment is disclosed in detail in the Combination Treatments section of this application.

In some aspects, the conjugates of the disclosure are administered to the subject prior to or after the administration of the additional therapeutic agent. In other aspects, the conjugates of the disclosure are administered to the subject concurrently with the additional therapeutic agent. In certain aspects, the conjugates of the disclosure and the additional therapeutic agent can be administered concurrently as a single composition in a pharmaceutically acceptable carrier. In other aspects, the conjugates of the disclosure and the additional therapeutic agent are administered concurrently as separate compositions.

In some aspects, a subject that can be treated with a conjugate of the present disclosure is a nonhuman animal such as a rat or a mouse. In some aspects, the subject that can be treated is a human.

### V. Methods of Preparing NeoDegraders and Compositions

### General Synthetic Methods and Intermediates

The compounds of the present disclosure can be prepared by one of ordinary skill in the art in light of the present disclosure and knowledge in the art, and/or by reference to the schemes shown below and the synthetic examples. Exemplary synthetic routes are set forth in schemes below and in Examples. It should be understood that the variables, (for example “R” groups) appearing in the following schemes and examples are to be read independently from those appearing elsewhere in the application. One of ordinary skill in the art would readily understand how the schemes and examples shown below illustrate the preparation of the compounds described herein.

### EXAMPLES

### General Synthetic Methods and Intermediates

The compounds of the present disclosure can be prepared by one of ordinary skill in the art in light of the present disclosure and knowledge in the art, and/or by reference to the schemes shown below and the synthetic examples. Exemplary synthetic routes are set forth in Schemes below and in Examples. It should be understood that the variables, (for example “R” groups) appearing in the following schemes and examples are to be read independently from those appearing elsewhere in the application. One of ordinary skill in the art would readily understand how the schemes and examples shown below illustrate the preparation of the compounds described herein.

Abbreviations used in the schemes generally follow conventions used in the art. Chemical abbreviations used in the specification and examples are defined as follows: “TCEP” for (tris(2-carboxyethyl)phosphine) and “EDTA” for ethylenediamninetetraacetic acid.

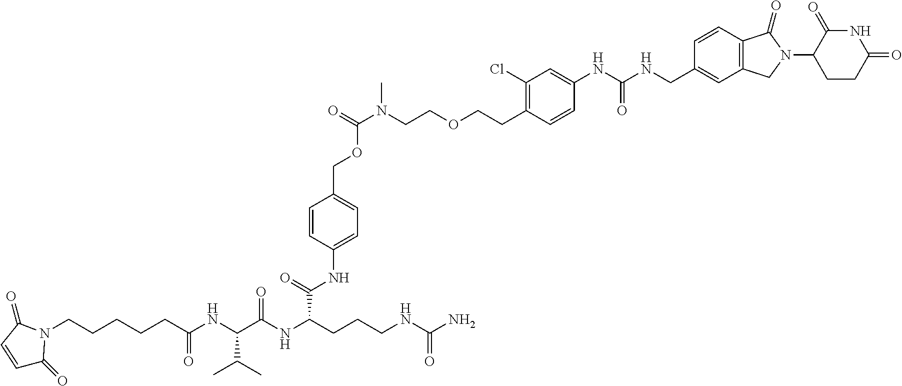 [SMILES: CC(C)[C@H](NC(=O)CCCCCN1C(=O)C=CC1=O)C(=O)N[C@@H](CCCNC(N)=O)C(=O)Nc1ccc(COC(=O)N(C)CCOCCc2ccc(NC(=O)NCc3ccc4c(c3)CN(C3CCC(=O)NC3=O)C4=O)cc2Cl)cc1]

### Example 1: Synthesis of Compound (III)

Compound (III) was prepared using the procedure described in WO2021/198965, which is incorporated herein by reference in its entirety.

### Example 2: Synthesis of Compounds (IV) and (IV-1)

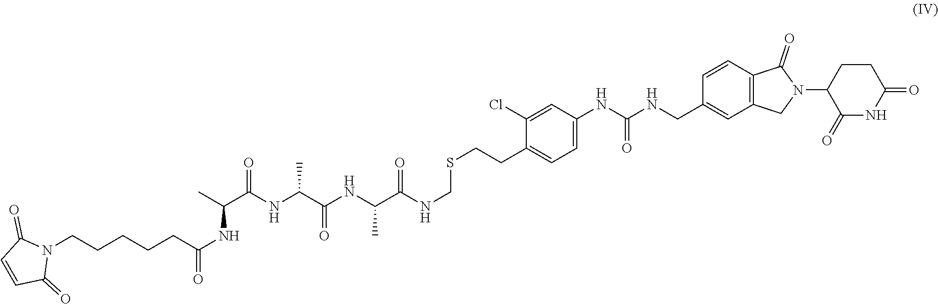 [SMILES: C[C@H](NC(=O)CCCCCN1C(=O)C=CC1=O)C(=O)N[C@H](C)C(=O)N[C@@H](C)C(=O)NCSCCc1ccc(NC(=O)NCc2ccc3c(c2)CN(C2CCC(=O)NC2=O)C3=O)cc1Cl]

Compound (IV) was prepared using the procedure described in WO2021/198965 which is incorporated herein by reference in its entirety.

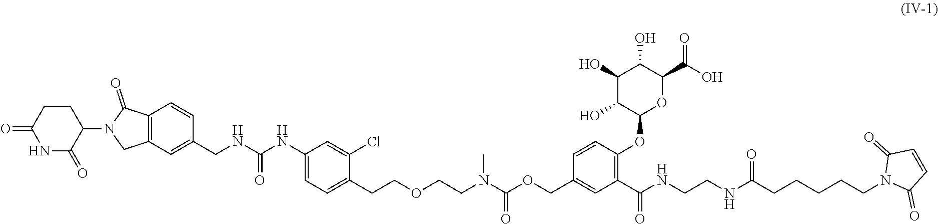 [SMILES: CN(CCOCCc1ccc(NC(=O)NCc2ccc3c(c2)CN(C2CCC(=O)NC2=O)C3=O)cc1Cl)C(=O)OCc1ccc(O[C@@H]2O[C@H](C(=O)O)[C@@H](O)[C@H](O)[C@H]2O)c(C(=O)NCCNC(=O)CCCCCN2C(=O)C=CC2=O)c1]

Compound (IV-1) was prepared using the procedure described in WO2022/254376, which is incorporated herein by reference in its entirety.

### Example 3: Preparation and Characterization of Compound (III)—Antibody CD56-A DAR3.7 Conjugate (Conjugate (V))

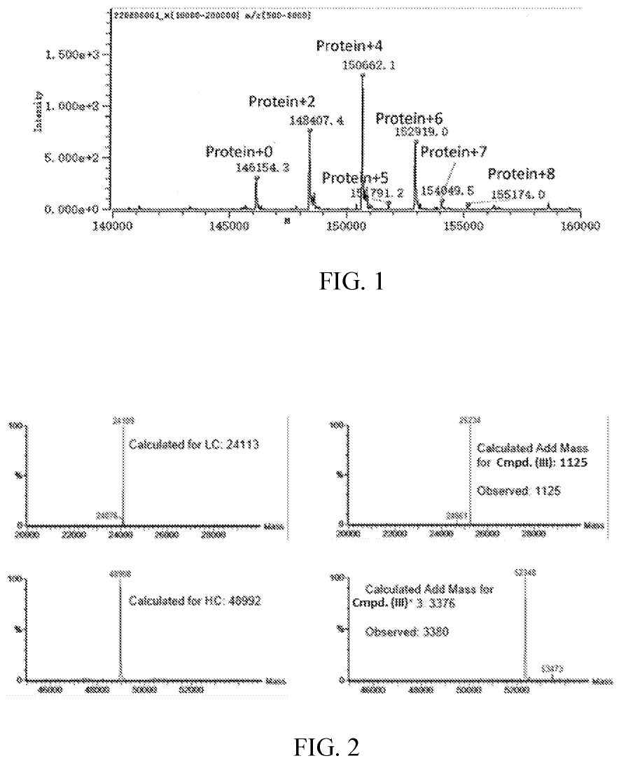

Antibody CD56-A at 10.7 mg/mL in 20 mM histidine, 250 mM sucrose pH 6.5 was reacted with 2.3 eq. TCEP for 1.5 h at 37 C. The partially reduced antibody was buffer exchanged into 50 mM EPPS, 5 mM EDTA pH 7.0 using NAP desalting columns. Then 3.20 mg/mL reduced antibody+12 eq. Compound (III) in 50 mM EPPS, 5 mM EDTA pH 7.0+10% DMA were allowed to react for 1 h at ambient temperature, then quenched with 0.01 volumes of 100 mM N-acetylcysteine. The resulting conjugate was purified using two rounds of gel filtration on NAP25 columns, eluting with 20 mM succinate, 8% sucrose, 0.01% Tween-20 pH 5.5. Concentration of the conjugate was carried out using 50K MWCO centrifugal concentrators. The conjugate was then sterile filtered through 0.22 um PVDF syringe filters. The final purified conjugate had a DAR of 3.7 by native SEC-MS, 4.45 mg/ml protein concentration, 99.6% monomer, and 2.3% unconjugated small molecule (FIG. 1). DAR was calculated using the weighted average of each peak intensity which is shown in Table 1.

TABLE 1
Weighted Average of Peaks in FIG. 1 SEC-MS

| Species | Mass | Intensity |
|---|---|---|
| 8-drug | 155174.0 | 54.203 |
| 7-drug | 154049.5 | 85.415 |
| 6-drug | 152919.0 | 655.624 |
| 5-drug | 151791.2 | 63.297 |
| 4-drug | 150662.1 | 1301.210 |
| 2-drug | 148407.4 | 770.376 |
| 0-drug | 146154.3 | 312.704 |

### Example 4: Preparation and Characterization of Compound (III)—Antibody CD56-A DAR8 Conjugate (Conjugate (VI))

Antibody CD56-A at 10.7 mg/mL in 20 mM histidine, 250 mM sucrose pH 6.5 was reacted with 10 eq. TCEP for 1.5 h at 37 C. The fully reduced antibody was buffer exchanged into 50 mM EPPS, 5 mM EDTA pH 7.0 using NAP desalting columns. Then 3.20 mg/mL reduced antibody+12 eq. Compound (III) in 50 mM EPPS, 5 mM EDTA pH 7.0+10% DMA were allowed to react for 1 h at ambient temperature, then quenched with 0.01 volumes of 100 mM N-acetylcysteine. The resulting conjugate was purified using two rounds of gel filtration on NAP25 columns, eluting with 20 mM succinate, 8% sucrose, 0.01% Tween-20 pH 5.5. Concentration of the conjugate was carried out using 50K MWCO centrifugal concentrators. The conjugate was then sterile filtered through 0.22 um PVDF syringe filters. The final purified conjugate had a DAR of 8 by reducing LC-MS, 3.16 mg/ml protein concentration, 98.1% monomer, and 0.6% unconjugated small molecule (FIG. 2). As shown in the figure, the conjugate has one linker-payload molecule attached to each light chain and three linker-payloads attached to each heavy chain, consistent with a DAR of 8.

### Example 5: Preparation and Characterization of Compound (III)—Antibody CD56-B DAR3.6 Conjugate (Conjugate (VII))

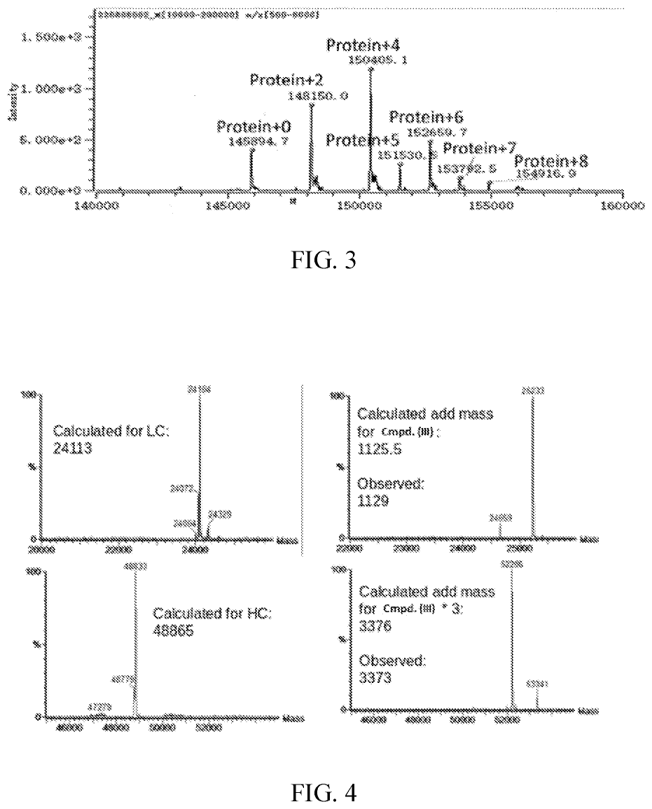

Antibody CD56-B at 9 mg/mL in 20 mM histidine, 250 mM sucrose pH 6.5 was reacted with 2.3 eq. TCEP for 1.5 h at 37 C. The reduced antibody was buffer exchanged into 50 mM EPPS, 5 mM EDTA pH 7.0 using NAP desalting columns. Then 3.20 mg/mL reduced antibody+12 eq. Compound (III) in 50 mM EPPS, 5 mM EDTA pH 7.0+10% DMA were allowed to react for 1 h at ambient temperature, then quenched with 0.01 volumes of 100 mM N-acetylcysteine. The resulting conjugate was purified using two rounds of gel filtration on NAP25 columns, eluting with 20 mM succinate, 8% sucrose, 0.01% Tween-20 pH 5.5. Concentration of the conjugate was carried out using 50K MWCO centrifugal concentrators. The conjugate was then sterile filtered through 0.22 um PVDF syringe filters. The final purified conjugate had a DAR of 3.6 by native SEC-MS, 4.1 mg/ml protein concentration, 100% monomer, and <2.1% unconjugated small molecule (FIG. 3). DAR was calculated using the weighted average of each peak intensity which is shown in Table 2.

TABLE 2
Weighted Average of Peaks in FIG. 3 SEC-MS

| Species | Mass | Intensity |
|---|---|---|
| 8-drug | 154916.9 | 81.369 |
| 7-drug | 153792.5 | 127.162 |
| 6-drug | 152659.7 | 488.112 |
| 5-drug | 151530.5 | 259.018 |
| 4-drug | 150405.1 | 1194.280 |
| 2-drug | 148150.0 | 847.745 |
| 0-drug | 145894.7 | 398.802 |

### Example 5-1: Preparation and Characterization of Compound (III)—Antibody CD56-B DAR8 Conjugate (Conjugate (VIII))

Antibody CD56-B at 9 mg/mL in 20 mM histidine, 250 mM sucrose pH 6.5 was reacted with 10 eq. TCEP for 1.5 h at 37 C. The fully reduced antibody was buffer exchanged into 50 mM EPPS, 5 mM EDTA pH 7.0 using NAP desalting columns. Then 3.20 mg/mL reduced antibody+12 eq. Compound (III) in 50 mM EPPS, 5 mM EDTA pH 7.0+10% DMA were allowed to react for 1 h at ambient temperature, then quenched with 0.01 volumes of 100 mM N-acetylcysteine. The resulting conjugate was purified using two rounds of gel filtration on NAP25 columns, eluting with 20 mM succinate, 8% sucrose, 0.01% Tween-20 pH 5.5. Concentration of the conjugate was carried out using 50K MWCO centrifugal concentrators. The conjugate was then sterile filtered through 0.22 um PVDF syringe filters. The final purified conjugate had a DAR of 8 by reducing LC-MS, 4.54 mg/ml protein concentration, 99% monomer, and 2.88% unconjugated small molecule (FIG. 4). As shown in the figure, the conjugate has one linker-payload molecule attached to each light chain and three linker-payloads attached to each heavy chain, consistent with a DAR of 8.

### Example 5-2: Preparation and Characterization of Compound (IV-1)—Antibody CD56-B DAR4.0 Conjugate (Conjugate IX)

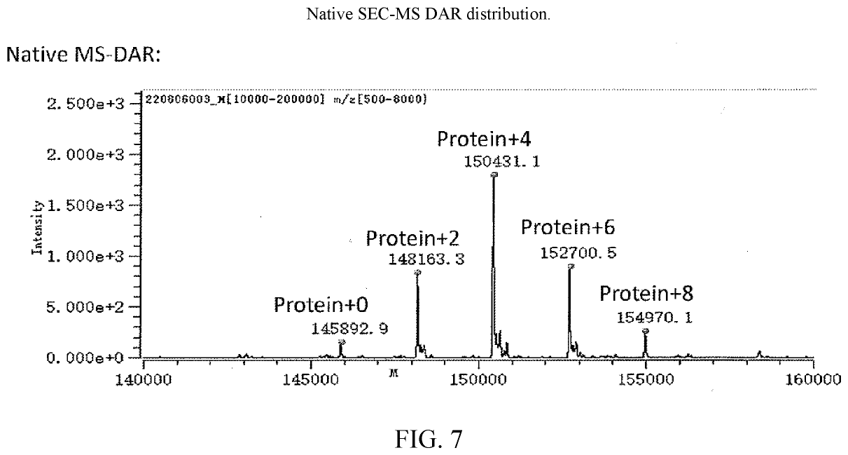

Antibody CD56-B at 6 mg/mL in 20 mM histidine, 250 mM sucrose pH 6.5 was reacted with 2.0 eq. TCEP for 2 h at 37° C. The reduced antibody was buffer exchanged into 50 mM EPPS, 5 mM EDTA pH 7.0 using Zeba desalting columns. Then 4.5 mg/mL reduced antibody+7 eq. Compound (IV-1) in 50 mM EPPS, 5 mM EDTA pH 7.0+10% DMA were allowed to react for 1 h at 22° C. The resulting conjugate was purified using two rounds of filtration using Zeba desalting columns, eluting with 20 mM succinate, 8% sucrose, 0.01% Tween-20 pH 5.5, 10% DMA, then with 20 mM succinate, 8% sucrose, 0.01% Tween-20 pH 5.5. Concentration of the conjugate was carried out using 50K MWCO centrifugal concentrators. The conjugate was then sterile filtered through 0.22 um PVDF syringe filters. The final purified conjugate had a DAR of 4.1 by native SEC-MS, 5.1 mg/mL protein concentration, 100% monomer, and <2.3% unconjugated small molecule (FIG. 7). DAR was calculated using the weighted average of each peak intensity which is shown in Table 2-1.

TABLE 2-1
Weighted Average of Peaks in FIG. 7 SEC-MS

| Species | Mass | Intensity |
|---|---|---|
| 8-drug | 154970.1 | 264.584 |
| 6-drug | 152700.5 | 897.198 |
| 4-drug | 150431.1 | 1800.210 |
| 2-drug | 148163.3 | 840.609 |
| 0-drug | 145892.9 | 158.371 |

### Example 5-3: Preparation and Characterization of Compound (IV)—Antibody CD56-B DAR4.0 Conjugate (Conjugate (X))

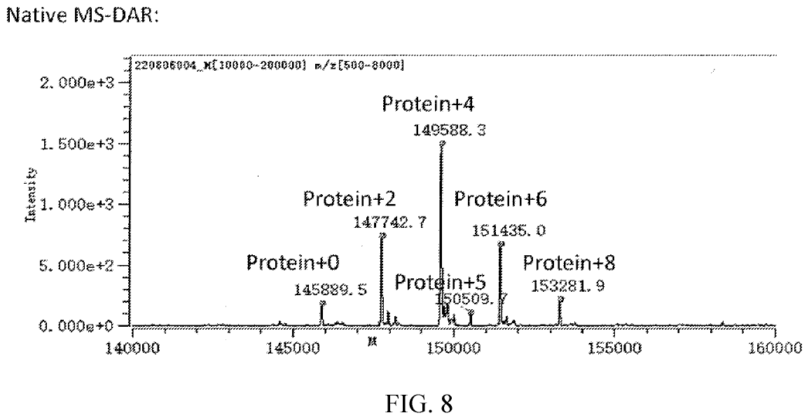

Antibody CD56-B at 6 mg/mL in 20 mM histidine, 250 mM sucrose pH 6.5 was reacted with 2.0 eq. TCEP for 2 h at 37° C. The reduced antibody was buffer exchanged into 50 mM EPPS, 5 mM EDTA pH 7.0 using Zeba desalting columns. Then 4.5 mg/mL reduced antibody+7 eq. Compound (IV) in 50 mM EPPS, 5 mM EDTA pH 7.0+10% DMA were allowed to react for 1 h at 22° C. The resulting conjugate was purified using two rounds of filtration using Zeba desalting columns, eluting with 20 mM succinate, 8% sucrose, 0.01% Tween-20 pH 5.5, 10% DMA, then with 20 mM succinate, 8% sucrose, 0.01% Tween-20 pH 5.5. Concentration of the conjugate was carried out using 50K MWCO centrifugal concentrators. The conjugate was then sterile filtered through 0.22 um PVDF syringe filters. The final purified conjugate had a DAR of 4.0 by native SEC-MS, 4.6 mg/ml protein concentration, 100% monomer, and <2.3% unconjugated small molecule (FIG. 8). DAR was calculated using the weighted average of each peak intensity which is shown in Table 2-2.

TABLE 2-2
Weighted Average of Peaks in FIG. 8 SEC-MS

| Species | Mass | Intensity |
|---|---|---|
| 8-drug | 153281.9 | 223.050 |
| 6-drug | 151435.0 | 681.889 |
| 5-drug | 150509.7 | 111.927 |
| 4-drug | 149588.3 | 1501.830 |
| 2-drug | 147742.7 | 747.757 |
| 0-drug | 145889.5 | 193.241 |

Other conjugates described herein can be prepared by the methods described above substituting the appropriate compounds and antibodies.

### Example 6: In Vitro Cytotoxicity Assay I

A cytotoxicity assay was performed to compare Compound (III)—antibody CD56-B conjugate DAR8 (Conjugate (VIII) and Compound (III)—antibody CD56-B conjugate DAR 3.6 (Conjugate (VII)), unconjugated antibody CD56-B, neoDegrader P1 (from WO2021/198965), CC-885, and a conjugate containing Compound (III) and a non-binding control antibody (Antibody-NBC) using a human SCLC cell line (NCI-H526) and a human neuroblastoma (NB) cell line (IMR32). Cell lines were harvested and plated into 96-well plates. After overnight incubation, the cells were treated with serial dilutions of Conjugate (VIII), Conjugate (VII), unconjugated antibody CD56-B, neoDegrader P1, Compound (III)—Antibody-NBC DAR3.9 conjugate (non-binding control conjugate), or CC-885 (Celgene) for 120 hours. The cell viability was determined by colorimetric WST-8 assay (Dojindo Molecular Technologies, Inc.). The OD₄₅₀ value was read using the SpectraMax M5 plate reader. The values were normalized for each cell line, and IC₅₀ values were calculated using the Prism software. As shown in Table 3, the Compound (III)—antibody CD56-B conjugate showed cytotoxic activity in the NCI-H526 and IMR32 cell lines and was more potent than the unconjugated antibody or neoDegrader P1.

TABLE 3
In Vitro Cytotoxicity IC50 Values (M)

| | NCI-H526 | IMR32 |
|---|---|---|
| Compound (III) - | 4.58E−12 | 2.42E−12 |
| Antibody CD56-B | | |
| DAR8 Conjugate | | |
| (Conjugate (VIII)) | | |
| Compound (III) - | 3.41E−11 | 1.68E−11 |
| Antibody CD56-B | | |
| DAR 3.6 Conjugate | | |
| (Conjugate (VII)) | | |
| Compound (IV-1)- | 1.53E−11 | 8.65E−12 |
| Antibody CD56-B | | |
| DAR 3.6 Conjugate | | |
| (Conjugate (IX)) | | |
| Antibody CD56-B | >2.00E−08 | >2.00E−08 |
| NeoDegrader P1 | 7.37E−09 | 6.85E−09 |
| CC-885 | 1.18E−10 | 1.45E−10 |
| Compound (III) - | >2.00E−08 | >2.00E−08 |
| Antibody-NBC DAR | | |
| 3.9 Conjugate | | |

### Example 7: In Vitro Cytotoxicity Assay II

A cytotoxicity assay was performed to compare Compound (III)— Antibody CD56-B DAR8 conjugate (Conjugate (VIII)) and Compound (III)— Antibody CD56-A DAR8 conjugate (Conjugate (VI)) using 5 small cell lung cancer (SCLC) cell lines (NCI-H526, NCI-H146, NCI-H82, NCI-H1048, NCI-H510A). Cell lines were harvested and plated into 96-well plates. After overnight incubation, the cells were treated with serial dilutions of Conjugate (VIII), Conjugate (VI), Compound (III)—Antibody-NBC DAR 3.9 conjugate (non-binding control conjugate), or CC-885 for 120 hours. The cell viability was determined by colorimetric WST-8 assay (Dojindo Molecular Technologies, Inc.). The OD₄₅₀ value was read using the SpectraMax M5 plate reader. The values were normalized for each cell line, and IC₅₀s were calculated using the Prism software.

As shown in Table 4, Conjugate (VIII) and Conjugate (VI) had similar growth inhibitory effects on all SCLC cell lines, with IC₅₀-values from 1.15×10⁻¹⁰ to 1.18×10⁻¹² M range. In the same panel of cell lines, treatment with the non-binding conjugate (Compound (III)—Antibody-NBC DAR 3.9) did not show SCLC growth inhibition. This demonstrates that the anti-proliferative activity of Conjugate (VIII) and Conjugate (VI) are specific and antigen-dependent.

TABLE 4
In Vitro Cytotoxicity IC50 Values (M)

| | | | Compound | |
|---|---|---|---|---|
| | | | (III)-Antibody- | |
| | Conjugate | Conjugate | NBC DAR 3.9 | |
| SCLC list | (VIII) | (VI) | Conjugate | CC-885 |
| NCI-H526 | 2.77E−11 | 5.48E−11 | >2.00E−08 | 1.77E−10 |
| NCI-H146 | 1.74E−11 | 3.21E−11 | >2.00E−08 | 5.27E−10 |
| NCI-H82 | 7.23E−11 | 1.15E−10 | >2.00E−08 | 6.03E−10 |
| NCI-H1048 | 1.18E−12 | 5.42E−12 | 1.27E−08 | 4.09E−11 |
| NCI-H510A | 3.96E−11 | 5.04E−11 | >2.00E−08 | 2.30E−10 |

### Example 8: In Vitro Cytotoxicity Assay III

A cytotoxicity assay was performed to compare Compound (III)—antibody CD56-B DAR8 (Conjugate (VIII)) and Compound (III)—antibody CD56-B DAR3.6 (Conjugate (VII)) using a human SCLC cell line (NCI-H526) and a human neuroblastoma (NB) cell line (IMR32). Cell lines were harvested and plated into 96-well plates. After overnight incubation, the cells were treated with serial dilutions of Conjugate (VIII), (Conjugate VII), Compound (III)—Antibody-NBC DAR 3.9 conjugate (non-binding control conjugate), or CC-885 for 120 hours. The cell viability was determined by colorimetric WST-8 assay (Dojindo Molecular Technologies, Inc.). The OD₄₅₀ value was read using the SpectraMax M5 plate reader. The values were normalized for each cell line, and IC₅₀ values were calculated using the Prism software.

As shown in Table 5, anti-proliferative effects were observed with Conjugate (VIII) and Conjugate (VII) in the NCI-H526 (SCLC) and IMR32 (NB) cell lines, with the former having a more robust effect. In contrast, the non-binding control conjugate, Compound (III)—Antibody-NBC DAR3.9 conjugate, had no cytotoxic effect against these cell lines.

TABLE 5
In Vitro Cytotoxicity IC50 Values (M)

| | | | Compound | |
|---|---|---|---|---|
| | | | (III)-Antibody- | |
| Cell Line | Conjugate | Conjugate | NBC DAR3.9 | |
| List | (VIII) | (VII) | Conjugate | CC-885 |
| NCI-H526 | 6.14E−12 | 1.74E−11 | >2.00E−08 | 1.28E−10 |
| IMR32 | 2.28E−12 | 6.49E−12 | >2.00E−08 | 9.94E−11 |

### Example 9: In Vitro Cytotoxicity Assay IV

A cytotoxicity assay was performed to check the anti-proliferative effect of Compound (III)—antibody CD56-B DAR8 conjugate (Conjugate (VIII)) in other cancer cell lines. One day prior to treatment with Conjugate (VIII) each tested cancer cell line was harvested and plated into 96-well plates. The cells were treated with Conjugate (VIII) for 5 days. Then cell viability was detected with the Cell Counting kit-8 (Dojindo) or CellTiter-Glo® reagent (Promega). The values were normalized to the untreated control for each cell line, and the IC₅₀s were calculated using Prism software.

As shown in Table 6, 3 sarcoma cell lines (Aska-SS, SJCRH30, and RH41), Conjugate (VIII) showed similar growth inhibitory effects, with IC₅₀ values from 8.09 to 2.02×10⁻¹¹M range. In the human neuroblastoma cell lines (KPNRTBM1, NB1, CHP134, and IMR32), Conjugate (VIII) also showed IC₅₀ values from 1.23×10⁻¹⁰ to 2.35×10⁻¹³ M range. Among them, KPNRTBM1 had a very sensitive anti-proliferative effect when treated with Conjugate (VIII). In the human NCIH660 neuroendocrine prostate cancer (NEPC) cell line, Conjugate (VIII) showed an IC₅₀ of 0.3 nM. In summary, Conjugate (VIII) showed potent efficacy against several tumor cell lines.

TABLE 6
In Vitro Cytotoxicity IC50 Values (M)

| Indication | Cell Line List | Conjugate (VIII) |
|---|---|---|
| Sarcoma | Aska-ss | 7.99E−11 |
| | SJCRH30 | 8.09E−11 |
| | RH41 | 2.02E−11 |
| NEPC | NCIH660 | 3.22E−10 |
| Neuroblastoma | KPNRTBM1 | 2.35E−13 |
| | NB1 | 2.49E−12 |
| | CHP134 | 1.49E−11 |
| | IMR32 | 1.23E−10 |

### Example 10: In Vivo Activity Study I

NCI-H660 human prostate cancer tumor cells (1×10⁷ cells in 0.1 mL) were subcutaneously inoculated into the flank of male BALB/c nude mice. The treatment was initiated when the mean tumor volume reached approximately 100-150 mm³. Mice were treated with a single dose of Compound (III)—antibody CD56-B DAR8 conjugate (Conjugate (VIII)), or Compound (IV)—antibody CD56-B DAR8 conjugate at a dose of 10 mg/kg (in 20 mM succinate, 8% sucrose, 0.01% Tween-20 pH 5.5) administered intravenously (IV). Tumor size and mouse body weight were measured twice per week. Tumor volume (mm³) was calculated with the following formula: (a×b²/2) where ‘b’ is the shortest axis and ‘a’ is the longest axis. Mice were taken off study when tumor volume for individual mice was >2,000 mm³ or Day 60, whichever came first.

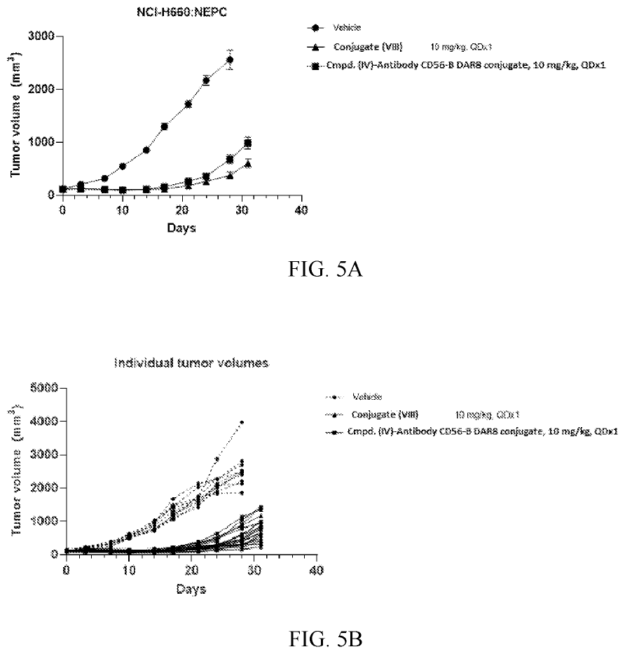

As shown in FIG. 5A, Conjugate (VIII) is observed to be slightly superior to Compound (IV)—antibody CD56-B DAR8 conjugate. FIG. 5B shows individual tumor volume over time for each dose group. FIG. 5C shows average body weight for mice in each dose group over the course of study. No other gross clinical abnormalities were observed during the study period.

### Example 11: In Vivo Activity Study II

NCI-H1048 human lung cancer cells (3×10⁶ cells in 0.1 mL) were subcutaneously inoculated into the flank of female BALB/c nude mice. The treatment was initiated when the mean tumor volume reached a mean of approximately 100 mm³ on day 12 post tumor cell inoculation. Mice were treated with a single dose of Compound (III)—antibody CD56-A 8 DAR conjugate (Conjugate (VI)) at a dose of 10 mg/kg (in 20 mM succinate, 8% sucrose, 0.01% Tween-20 pH 5.5) administered intravenously (IV). Tumor size and mouse body weight were measured twice per week. Tumor volume (mm³) was calculated with the following formula: (a×b²/2) where ‘b’ is the shortest axis and ‘a’ is the longest axis. Mice were taken off study when tumor volume for individual mice was >2,000 mm³ or day 60, whichever came first.

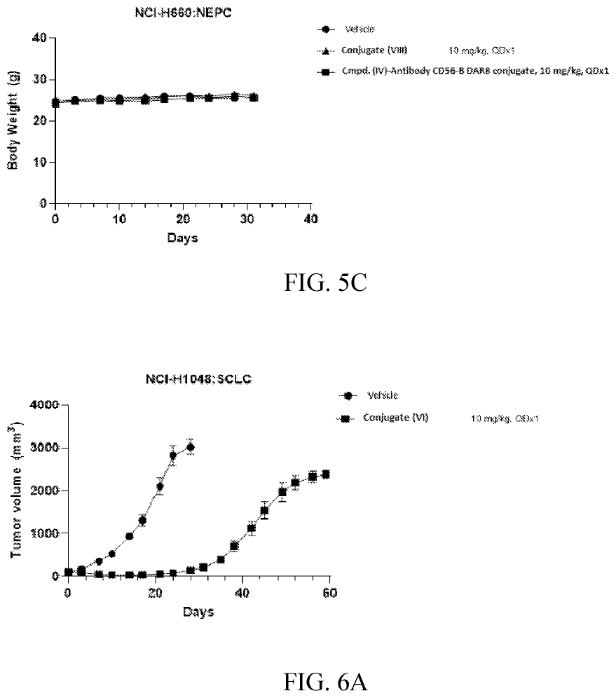

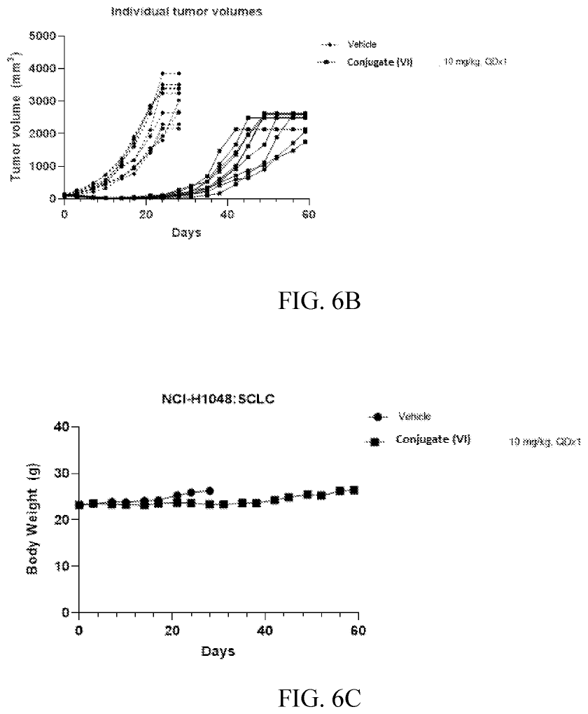

As shown in FIG. 6A, Conjugate (VI) showed significant antitumor activity with the mean tumor size of 73 mm³ (TGI 97.5%, P<0.001 vs. control) on day 36 post tumor inoculation. FIG. 6B shows individual tumor volume over time for each dose group. FIG. 6C shows average body weight for mice in each dose group over course of study. No other gross clinical abnormalities were observed during the study period.

### Example 12: In Vivo Activity Study III

IMR32 human neuroblastoma cancer tumor cells (1×10⁷ cells in 0.1 mL) were subcutaneously inoculated into the flank of female BALB/c nude mice. The treatment was initiated when the mean tumor volume reached approximately 125 mm³. Mice were treated with a single dose of Compound (III)—antibody CD56-B DAR8 conjugate (Conjugate (VIII)) at dose of 3 mg/kg or Compound (IV-1)—antibody CD56-B DAR3.64 conjugate (Conjugate (IX)) at a dose of 6.6 mg/kg (in 20 mM succinate, 8% sucrose, 0.01% Tween-20 pH 5.5) administered intravenously (IV). Conjugate (IX) dosing matches Conjugate (VIII) payload dosing). Tumor size and mouse body weight were measured twice per week. Tumor volume (mm³) was calculated with the following formula: (a×b²/2) where ‘b’ is the shortest axis and ‘a’ is the longest axis. Mice were taken off study when tumor volume for individual mice is >2,000 mm³ or Day 60, whichever came first.

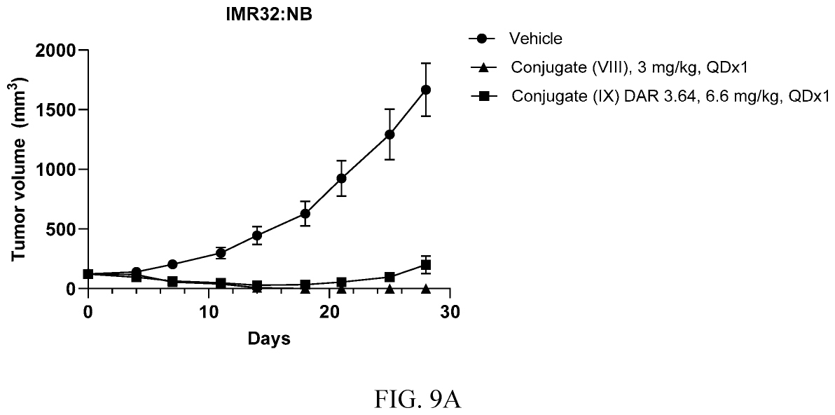

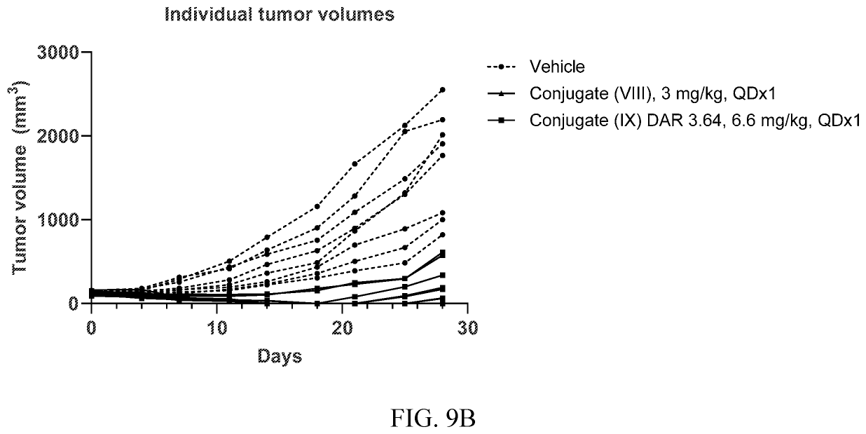

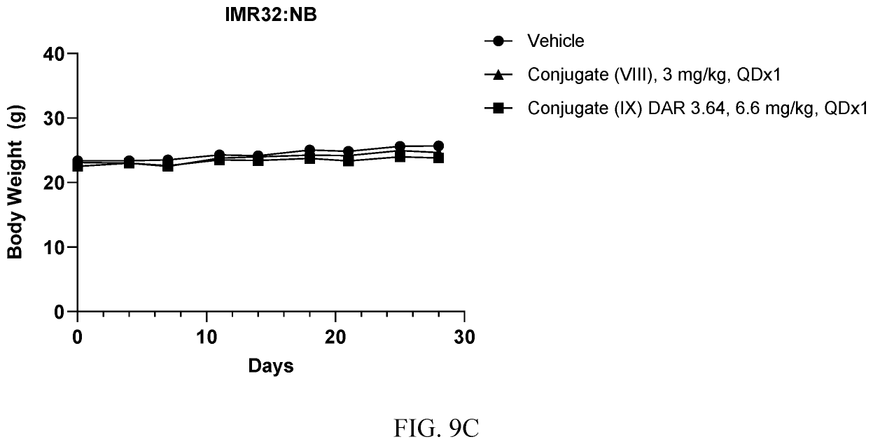

As shown in FIGS. 9A-9C, Conjugate (VIII) and Conjugate (IX) showed significant antitumor activity. Conjugate (VIII) at the dose level of 3 mg/kg and Conjugate (IX) at the dose level of 6.6 mg/kg showed significant antitumor activity with the mean tumor volume of 0 mm³ (TGI=100%, P<0.001 vs. control) and 104 mm³ (TGI=94.1%, P<0.001 vs. control) on day 28 post dosing, respectively (FIG. 9A). FIG. 9B shows individual tumor volume over time for each dose group. FIG. 9C shows average body weight for mice in each dose group over course of study. No other gross clinical abnormalities were observed during the study period.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A conjugate of formula (I) or formula (II): 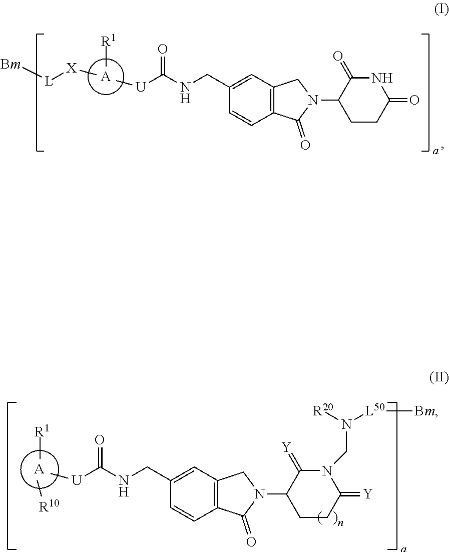
or a pharmaceutically acceptable salt thereof, wherein:
a is an integer from 1 to 10;
n is 0 or 1;
A is phenyl or a C₄-C₁₀cycloalkyl ring;
U is selected from NH, O, S, and CF₂;
R¹ is independently selected from hydrogen and halo;
R¹⁰ is selected from —CH₃, —C(O)R³⁰, —N(R⁴⁰)₂, —(CH₂)_{n′}OH, —(CH₂)_{n′}N(R⁴⁰)₂, —(CH₂)_{n′}Q(CH₂)_{m′}OH, —(CH₂)_{n′}Q(CH₂)_{m′}SH, and —(CH₂)_{n′}Q(CH₂)_{m′}N(R⁴⁰)₂; wherein:
R³⁰ is hydrogen or C₁-C₆alkyl;
each R⁴⁰ is independently hydrogen or C₁-C₆alkyl;
Q is O, S, or NR⁴⁰;
n′ is 1-6; and
m′ is 2-5;
R²⁰ is selected from hydrogen, —(CH₂CH₂O)_{v′}—CH₃, C₂-C₆alkenyl, C₁-C₆alkyl; C₂-C₆alkynyl, benzyl, C₃-C₆cycloalkyl, and C₃-C₆cycloalkyl(C₁-C₃alkyl), wherein v′ is from 1 to 24;
X is selected from —NR²⁰⁰—, =C(CH₃)—, -Q′—(CH₂)_{n′}—, and -Q′(CH₂)_{m″}Q″(CH₂)_{n″}—;
wherein:
Q′ and Q″ are each independently O, S, or N(R²⁰⁰)ᵥ, wherein:
v is 1 or 2;
each R²⁰⁰ is independently hydrogen or C₁-C₆alkyl;
n″ is an integer from 1 to 6; and
m″ is an integer from 2 to 6;
wherein the left side of each group is attached to L and the right side is attached to A;
provided that when X is NH or -Q′—(CH₂)_{n′}—, R¹ is halo;
each Y is independently S or O;
L is a cleavable linker or non-cleavable linker;
L⁵⁰ is a cleavable linker;
and
Bm is a binding moiety that is capable of specifically binding to CD56.

2. The conjugate of claim 1, wherein the binding moiety is an antibody or an antigen-binding fragment thereof.

3. The conjugate of claim 1 or 2, or a pharmaceutically acceptable salt thereof, wherein a is an integer from 2 to 8.

4. The conjugate of any one of claims 1 to 3, which is a conjugate of formula (I) or a pharmaceutically acceptable salt thereof, wherein L is a non-cleavable linker.

5. The conjugate of claim 4, or a pharmaceutically acceptable salt thereof, wherein L is selected from the group consisting of 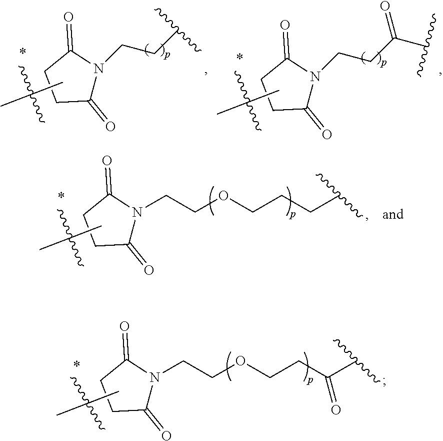
wherein:
p is an integer from 1 to 10; [symbol]
is the point of attachment to X; and [symbol]
is the point of attachment to the binding moiety.

6. The conjugate of claim 5, or a pharmaceutically acceptable salt thereof, wherein L is

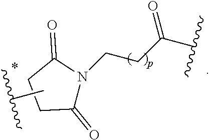

7. The conjugate of claim 6, or a pharmaceutically acceptable salt thereof, wherein p is 5.

8. The conjugate of any one of claims 1 to 3, which is a conjugate of formula (I) or a pharmaceutically acceptable salt thereof, wherein L is a cleavable linker.

9. The conjugate of claim 8, or a pharmaceutically acceptable salt thereof, wherein the cleavable linker is cleavable by a protease.

10. The conjugate of claim 8 or 9, or a pharmaceutically acceptable salt thereof, wherein L is selected from the group consisting of 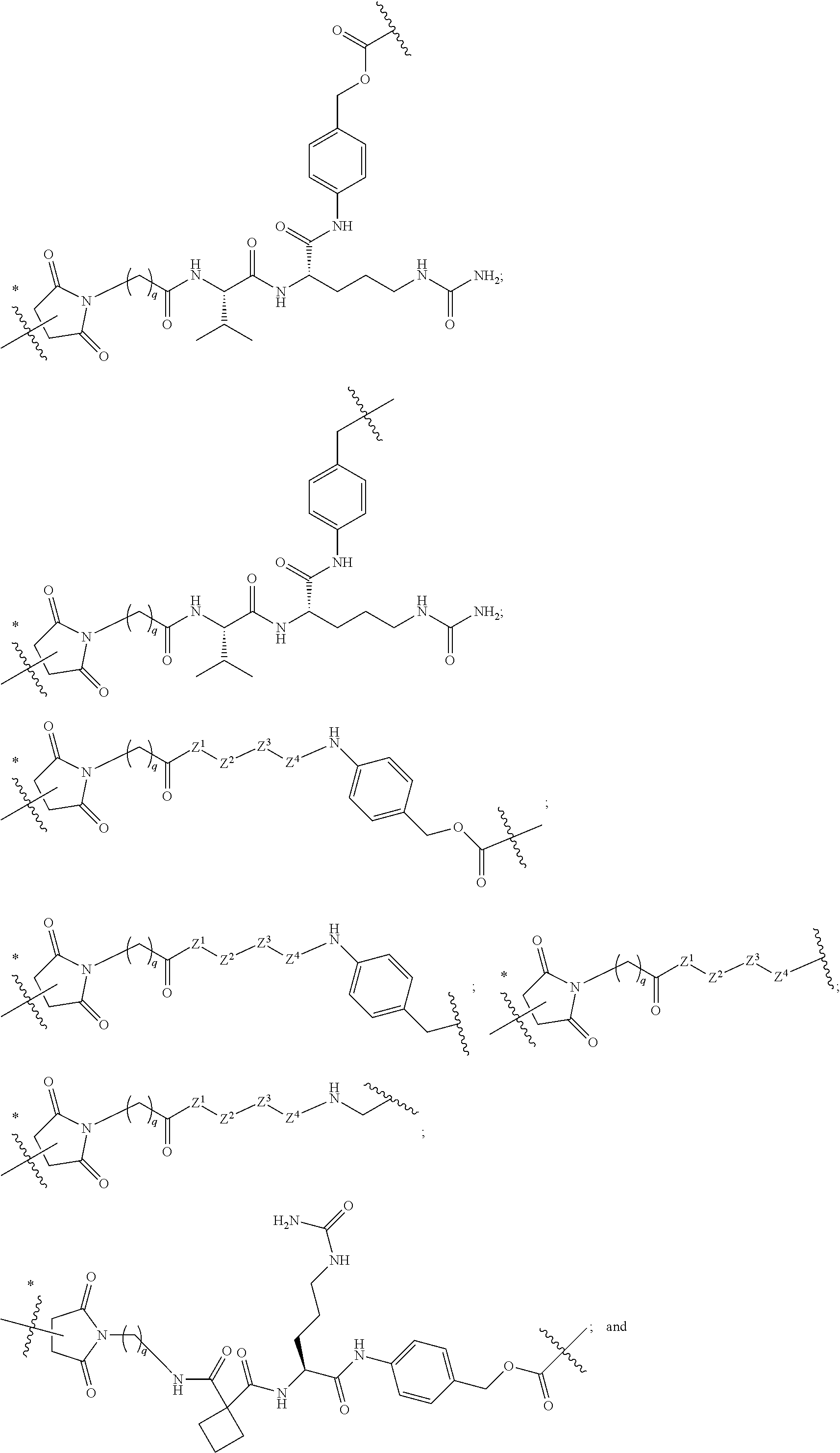 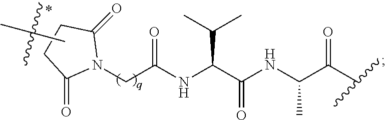
wherein:
q is an integer from 2 to 10;
Z¹, Z², Z³, and Z⁴ are each independently absent or a naturally-occurring amino acid residue in the L- or D-configuration, provided that at least two of Z¹, Z², Z³, and Z⁴ are amino acid residues; [symbol]
is the point of attachment to X; and [symbol]
is the point of attachment to the binding moiety.

11. The conjugate of claim 10, or a pharmaceutically acceptable salt thereof, wherein Z¹, Z², Z³, and Z⁴ are independently absent or selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, and Z⁴ are amino acid residues.

12. The conjugate of claim 10 or 11, or a pharmaceutically acceptable salt thereof, wherein:
Z¹ is absent or glycine;
Z² is absent or selected from the group consisting of L-glutamine, D-glutamine, L-glutamic acid, D-glutamic acid, L-aspartic acid, D-aspartic acid, L-alanine, D-alanine, and glycine;
Z³ is selected from the group consisting of L-valine, D-valine, L-alanine, D-alanine, L-phenylalanine, D-phenylalanine, and glycine; and
Z⁴ is selected from the group consisting of L-alanine, D-alanine, L-citrulline, D-citrulline, L-asparagine, D-asparagine, L-lysine, D-lysine, L-phenylalanine, D-phenylalanine, and glycine.

13. The conjugate of any one of claims 10 to 12, or a pharmaceutically acceptable salt thereof, wherein L is

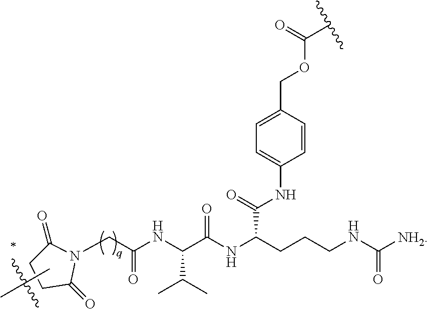

14. The conjugate of claim 13, or a pharmaceutically acceptable salt thereof, wherein q is 5.

15. The conjugate of claim 8, or a pharmaceutically acceptable salt thereof, wherein L is a bioreducible linker.

16. The conjugate of claim 15, wherein L is selected from the group consisting of 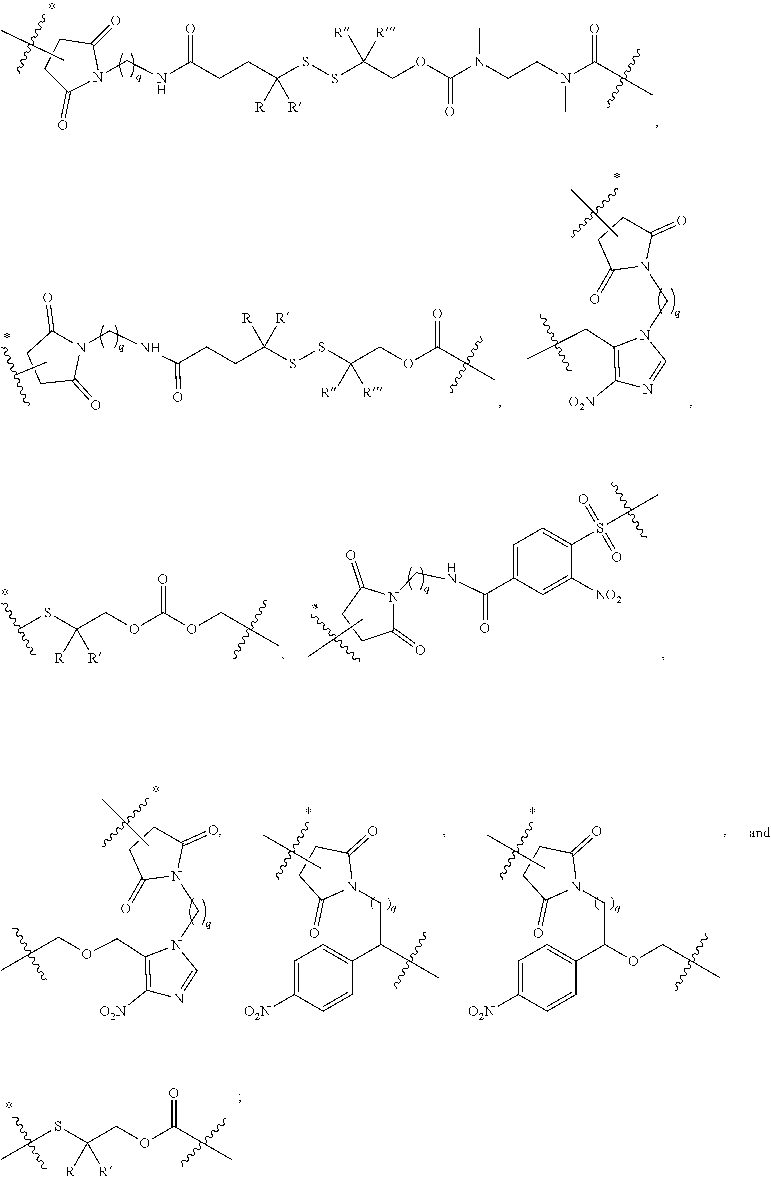
wherein:
q is an integer from 2 to 10;
R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring; [symbol]
is the point of attachment to X; and [symbol]
is the point of attachment to the binding moiety.

17. The conjugate of claim 8, or a pharmaceutically acceptable salt thereof, wherein L is an acid cleavable linker.

18. The conjugate of claim 17, or a pharmaceutically acceptable salt thereof, wherein L is selected from the group consisting of 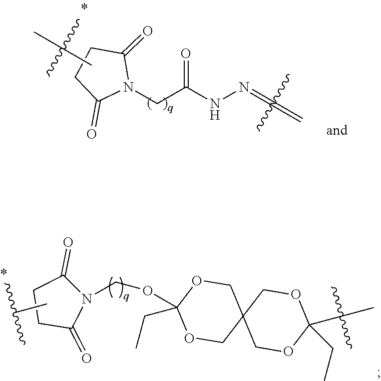
wherein:
q is an integer from 2 to 10; [symbol]
is the point of attachment to X; and [symbol]
is the point of attachment to the binding moiety.

19. The conjugate of claim 8, or a pharmaceutically acceptable salt thereof, wherein L is a click-to-release linker.

20. The conjugate of claim 19, or a pharmaceutically acceptable salt thereof, wherein L is selected from 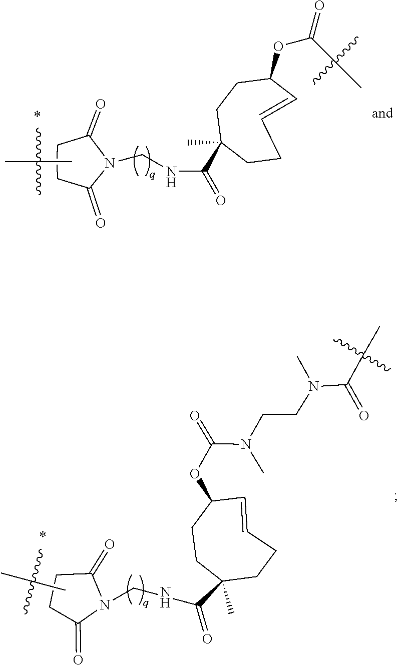
wherein:
q is an integer from 2 to 10; [symbol]
is the point of attachment to X; and [symbol]
is the point of attachment to the binding moiety.

21. The conjugate of claim 8, or a pharmaceutically acceptable salt thereof, wherein L is a pyrophosphatase cleavable linker.

22. The conjugate of claim 21, or a pharmaceutically acceptable salt thereof, wherein L is 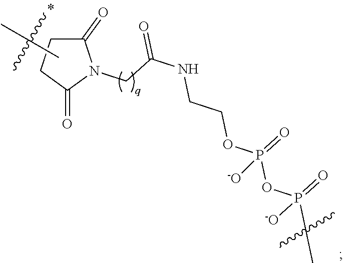
wherein:
q is an integer from 2 to 10; [symbol]
is the point of attachment to X; and [symbol]
is the point of attachment to the binding moiety.

23. The conjugate of claim 8, or a pharmaceutically acceptable salt thereof, wherein L is a beta-glucuronidase cleavable linker.

24. The conjugate of claim 23, or a pharmaceutically acceptable salt thereof, wherein L is selected from 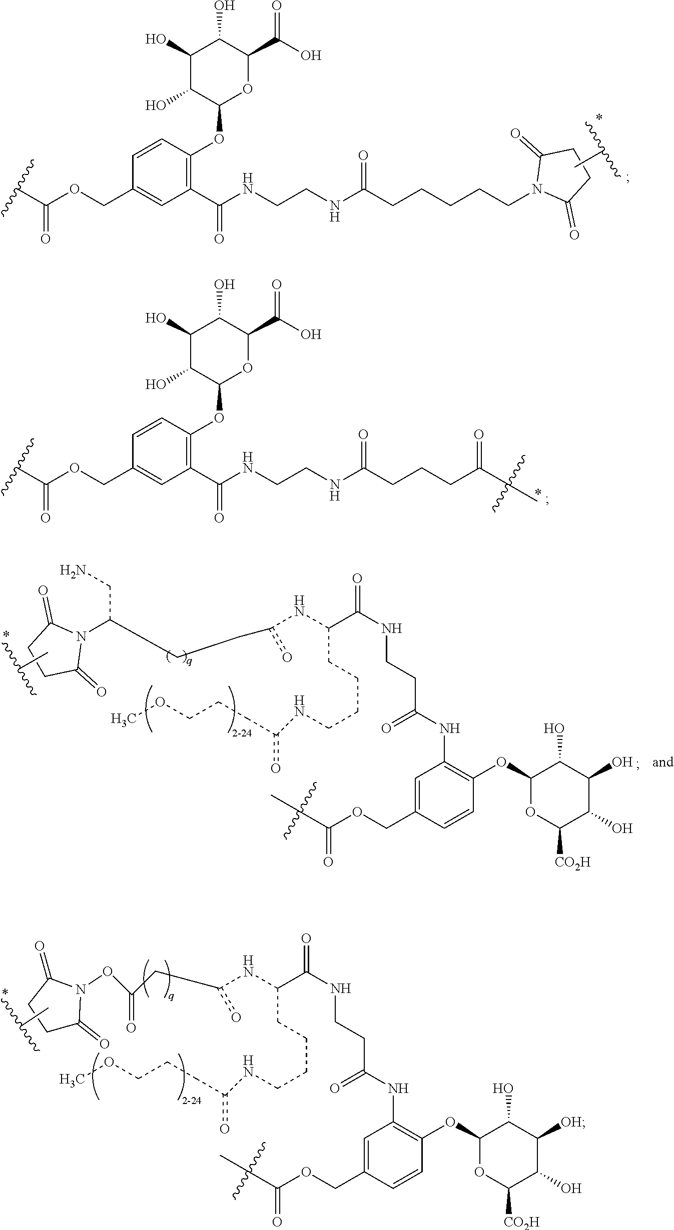
wherein:
q is an integer from 2 to 10; [symbol]
is absent or a bond; [symbol]
is the point of attachment to X; and [symbol]
is the point of attachment to the binding moiety.

25. The conjugate of claim 24, or a pharmaceutically acceptable salt thereof, wherein L is

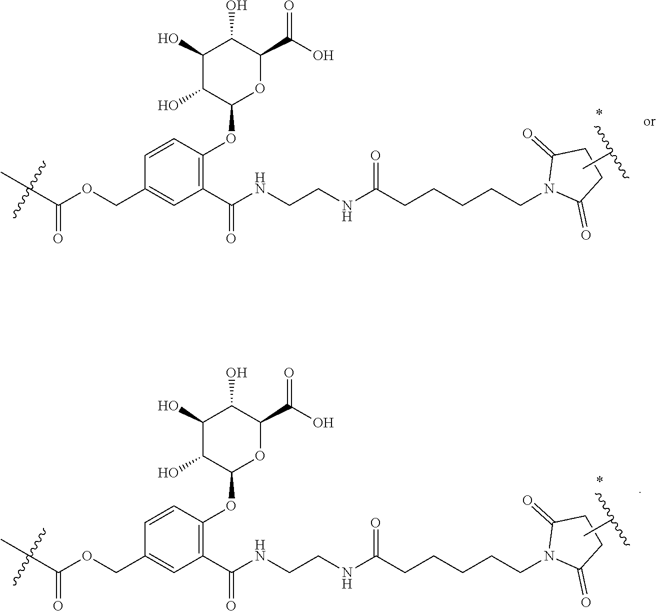

26. The conjugate of any one of claims 1 to 25, which is a conjugate of formula (I), or a pharmaceutically acceptable salt thereof, wherein:
A is phenyl;
U is NH;
R¹ is halo; and
X is —N(R²⁰⁰)ᵥ(CH₂)_{m″}O(CH₂)_{n″}—; wherein:
v is 1;
m″ and n″ are 2; and
R²⁰⁰ is methyl.

27. The conjugate of any one of claims 1 to 25, which is a conjugate of formula (I), or a pharmaceutically acceptable salt thereof, wherein:
A is phenyl;
U is NH;
R¹ is halo; and
X is —N(R²⁰⁰)ᵥ(CH₂)_{m″}O(CH₂)_{n″}—; wherein:
v is 2;
m″ and n″ are 2; and
each R²⁰⁰ is methyl.

28. The conjugate of any one of claims 1 to 25, which is a conjugate of formula (I), or a pharmaceutically acceptable salt thereof, wherein:
A is phenyl;
U is NH;
R¹ is halo; and
X is —O(CH₂)_{n″}—; wherein:
n″ is 2.

29. The conjugate of any one of claims 1 to 25, which is a conjugate of formula (I), or a pharmaceutically acceptable salt thereof, wherein:
A is phenyl;
U is NH;
R¹ is halo; and
X is —S(CH₂)_{n″}—; wherein:
n″ is 2.

30. The conjugate of any one of claims 1 to 25, which is a conjugate of formula (I), or a pharmaceutically acceptable salt thereof, wherein:
A is phenyl;
U is NH;
R¹ is hydrogen; and
X is —NR²⁰⁰—; wherein:
R²⁰⁰ is methyl.

31. The conjugate of any one of claims 1 to 25, which is a conjugate of formula (I), or a pharmaceutically acceptable salt thereof, wherein:
A is phenyl;
U is NH;
R¹ is halo; and
X is —NR²⁰⁰—; wherein:
R²⁰⁰ is hydrogen.

32. The conjugate of any one of claims 1 to 25, which is a conjugate of formula (I), or a pharmaceutically acceptable salt thereof, wherein:
A is phenyl;
U is NH;
R¹ is halo; and
X is —NR²—; wherein:
R² is hydrogen.

33. The conjugate of any one of claims 1 to 25, which is a conjugate of formula (I), or a pharmaceutically acceptable salt thereof, wherein:
A is phenyl;
U is NH;
R¹ is hydrogen; and
X is —C(CH₃)=.

34. The conjugate of any one of claims 1 to 25, which is a conjugate of formula (I), or a pharmaceutically acceptable salt thereof, wherein:
A is a C₄-C₁₀cycloalkyl ring;
U is NH;
R¹ is hydrogen; and
X is —N(R²⁰⁰)(CH₂)ₘO(CH₂)ₙ—; wherein:
n″ is 1;
m″ is 2; and
R²⁰⁰ is methyl.

35. The conjugate of any one of claims 1 to 3 which is a conjugate of formula (II), or a pharmaceutically acceptable salt thereof, wherein the cleavable linker is cleavable by a protease.

36. The conjugate of claim 35, wherein L⁵⁰ is selected from the group consisting of 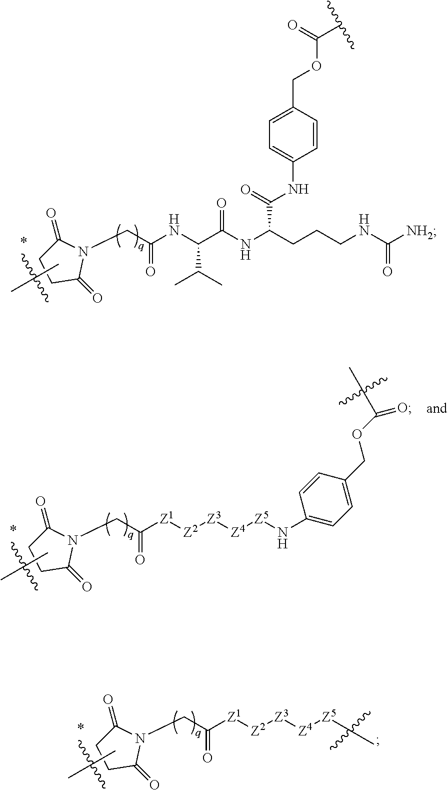
wherein:
q is from 2 to 10;
Z¹, Z², Z³, Z⁴, and Z⁵ are each independently absent or a naturally-occurring amino acid residue in the L- or D-configuration, provided that at least two of Z¹, Z², Z³, Z⁴, and Z⁵ are amino acid residues; [symbol]
is the point of attachment to the parent molecular moiety; and [symbol]
is the point of attachment to the binding moiety.

37. The conjugate of claim 36, or a pharmaceutically acceptable salt thereof, wherein Z¹, Z², Z³, Z⁴, and Z⁵ are independently absent or selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, Z⁴, and Z⁵ are amino acid residues.

38. The conjugate of claim 36 or 37, or a pharmaceutically acceptable salt thereof, wherein:
Z¹ is absent or glycine;
Z² is absent or selected from the group consisting of L-glutamine, D-glutamine, L-glutamic acid, D-glutamic acid, L-aspartic acid, D-aspartic acid, L-alanine, D-alanine, and glycine;
Z³ is selected from the group consisting of L-valine, D-valine, L-alanine, D-alanine, L-phenylalanine, D-phenylalanine, and glycine;
Z⁴ is selected from the group consisting of L-citrulline, D-citrulline, L-asparagine, D-asparagine, L-lysine, D-lysine, L-phenylalanine, D-phenylalanine, and glycine; and
Z⁵ is absent or glycine.

39. The conjugate of any one of claim 36 to 38, or a pharmaceutically acceptable salt thereof, wherein L⁵⁰ is

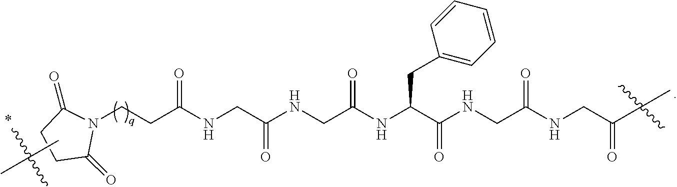

40. The conjugate of claim 39, wherein q is 4.

41. The conjugate of any one of claims 1 to 3 which is a conjugate of formula (II), or a pharmaceutically acceptable salt thereof, wherein L⁵⁰ is a bioreducible linker.

42. The conjugate of claim 41, or a pharmaceutically acceptable salt thereof, wherein L⁵⁰ is selected from the group consisting of 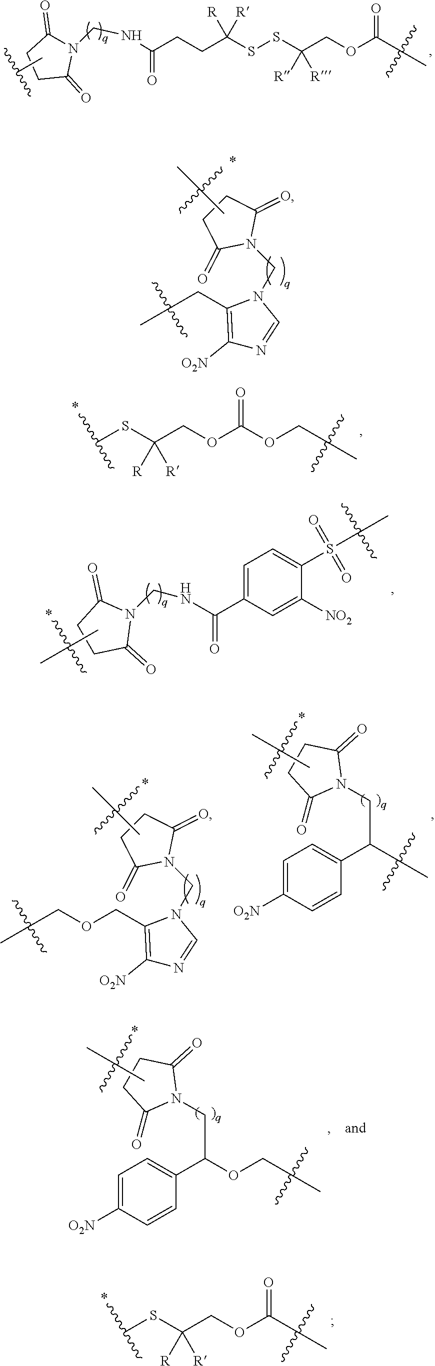
wherein:
q is from 2 to 10;
R, R′, R″, and R′″ are each independently selected from hydrogen, C₁-C₆alkoxyC₁-C₆alkyl, (C₁-C₆alkyl)₂NC₁-C₆alkyl, and C₁-C₆alkyl, or, two geminal R groups, together with the carbon atom to which they are attached, can form a cyclobutyl or cyclopropyl ring; [symbol]
is the point of attachment to the parent molecular moiety; and [symbol]
is the point of attachment to the binding moiety.

43. The conjugate of claim 41 or 42, or pharmaceutically acceptable salt thereof, wherein L⁵⁰ is

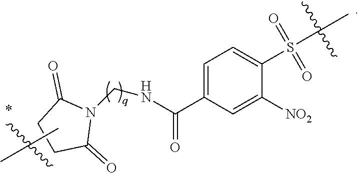

44. The conjugate of claim 43, or a pharmaceutically acceptable salt thereof, wherein q is 2.

45. The conjugate of any one of claims 1 to 3 which is a conjugate of formula (II), or a pharmaceutically acceptable salt thereof, wherein L⁵⁰ is a click-to-release linker.

46. The conjugate of claim 45, or a pharmaceutically acceptable salt thereof, wherein L⁵⁰ is 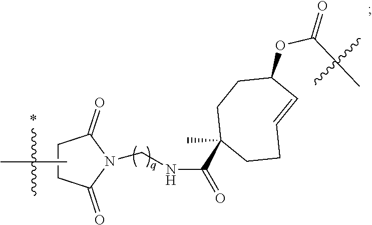
wherein:
q is from 2 to 10; [symbol]
is the point of attachment to the parent molecular moiety; and [symbol]
is the point of attachment to the binding moiety.

47. The conjugate of any one of claims 1 to 3 which is a conjugate of formula (II), or a pharmaceutically acceptable salt thereof, wherein L⁵⁰ is a beta-glucuronidase cleavable linker.

48. The conjugate of claim 47, or a pharmaceutically acceptable salt thereof, wherein L⁵⁰ is selected from 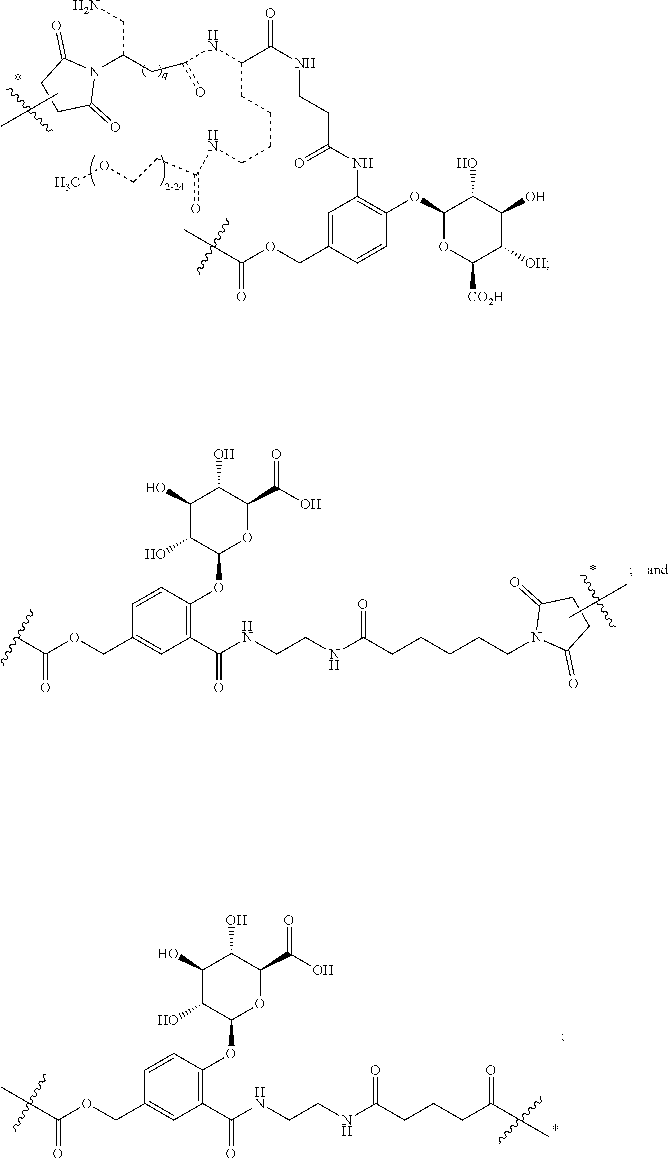
wherein:
q is from 2 to 10; [symbol]
is absent or a bond; [symbol]
is the point of attachment to the parent molecular moiety; and [symbol]
is the point of attachment to the binding moiety.

49. The conjugate of any one of claims 1 to 48, or a pharmaceutically acceptable salt thereof, wherein a is from 3 to 4.

50. The conjugate of any one of claims 1 to 48, or a pharmaceutically acceptable salt thereof, wherein a is 8.

51. The conjugate of any one of claims 1 to 50, or a pharmaceutically acceptable salt thereof, wherein the binding moiety comprises a variable heavy chain (VH) complementarity determining region (CDR) 1 comprising the amino acid sequence of SEQ ID NO:2, a VH CDR2 comprising the amino acid sequence of SEQ ID NO:3, a VH CDR3 comprising the amino acid sequence of SEQ ID NO:4, a variable light chain (VL) CDR1 comprising the amino acid sequence of SEQ ID NO:5, a VL CDR2 comprising the amino acid sequence of SEQ ID NO:6, and a VL CDR3 comprising the amino acid sequence of SEQ ID NO:7.

52. The conjugate of any one of claims 1 to 51, wherein the binding moiety comprises a VH comprising the amino acid sequence of SEQ ID NO:8 and/or a VL comprising the amino acid sequence of SEQ ID NO:9.

53. The conjugate of any one of claims 2 to 52, wherein the antibody or antigen-binding fragment thereof is an IgG antibody or an antigen-binding fragment thereof, optionally an IgG1 antibody or antigen-binding fragment thereof.

54. The conjugate of any one of claims 2 to 53, wherein the antibody or antigen-binding fragment thereof comprises (i) an N297A mutation according to EU numbering and/or (ii) a LALA (L234A and L235A) mutation according to EU numbering.

55. The conjugate of any one of claims 2 to 54, wherein the antibody or antigen-binding fragment thereof comprises a constant region comprising an engineered cysteine or a constant region comprising the amino acid sequence of any one of SEQ ID NOs:14-20.

56. The conjugate of any one of claims 1 to 55, wherein the binding moiety comprises (i) a heavy chain comprising the amino acid sequence of SEQ ID NO:10 and/or a light chain comprising the amino acid sequence of SEQ ID NO:11 or (ii) a heavy chain comprising the amino acid sequence of SEQ ID NO:12 and/or a light chain comprising the amino acid sequence of SEQ ID NO:11.

57. A conjugate which is: 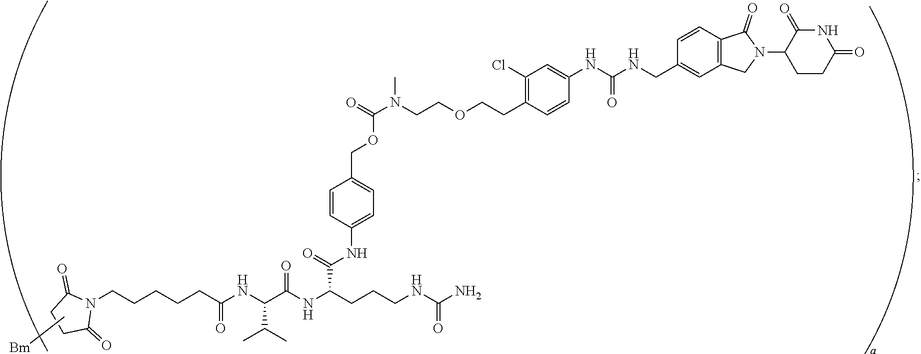
wherein Bm is antibody CD56-B and a is 3-8, optionally 3, 4, or 8.

58. A composition comprising at least one conjugate of any one of claims 1 to 57, wherein the average number of neodegraders per Bm is 3-8, optionally wherein the average number of neodegraders per Bm is about 3 to about 4 or wherein the average number of neodegraders per Bm is about 8.

59. A method of treating cancer in a subject in need thereof, the method comprising administering to the subject a pharmaceutically acceptable amount of a conjugate or composition of any of claims 1 to 58, or a pharmaceutically acceptable salt thereof.

60. The method of claim 59, wherein the cancer is CD56-positive cancer.

61. The method of claim 59 or 60, wherein the cancer is a neuroendocrine cancer (optionally wherein the neuroendocrine cancer is a neuroblastoma or neuroendocrine prostate cancer), a lung cancer (optionally wherein the lung cancer is a small cell lung cancer (SLCL)), or a sarcoma.

62. The method of claim 59, further comprising administering to the subject a pharmaceutically acceptable amount of an additional agent prior to, after, or simultaneously with the conjugate or composition of any one of claims 1 to 58, or a pharmaceutically acceptable salt thereof.

63. The method of claim 62, wherein the additional agent is a cytotoxic agent or an immune response modifier.

64. The method of claim 63, wherein the immune response modifier is a checkpoint inhibitor.

65. The method of claim 64, wherein the checkpoint inhibitor comprises a PD-1 inhibitor, a PD-L1 inhibitor, a CTLA-4 inhibitor, a TIM3 inhibitor, and/or a LAG-3 inhibitor.

66. Use of the conjugate or composition of any one of claims 1 to 58 in the preparation of a medicament for use in the method of any one of claims 59 to 65.


SEQUENCE LISTING (19 sequences)

SEQ ID NO: 1 - protein, 499 aa, Homo sapiens
  source 1..499: note = Human GSPT1
MELSEPIVENGETEMSPEESWEHKEEISEAEPGGGSLGDGRPPEESAHEMMEEEEEIPKPKSVVAPPGAPKKEHVNVVFIGHVDAGKSTIGGQIMYLTGMVDKRTLEKYEREAKEKNRETWYLSWALDTNQEERDKGKTVEVGRAYFETEKKHFTILDAPGHKSFVPNMIGGASQADLAVLVISARKGEFETGFEKGGQTREHAMLAKTAGVKHLIVLINKMDDPTVNWSNERYEECKEKLVPFLKKVGFNPKKDIHFMPCSGLTGANLKEQSDFCPWYIGLPFIPYLDNLPNFNRSVDGPIRLPIVDKYKDMGTVVLGKLESGSICKGQQLVMMPNKHNVEVLGILSDDVETDTVAPGENLKIRLKGIEEEEILPGFILCDPNNLCHSGRTFDAQIVIIEHKSIICPGYNAVLHIHTCIEEVEITALICLVDKKSGEKSKTRPRFVKQDQVCIARLRTAGTICLETFKDFPQMGRFTLRDEGKTIAIGKVLKLVPEKD

SEQ ID NO: 2 - protein, 5 aa, synthetic construct
  source 1..5: note = VH-CDR1
SFGMH

SEQ ID NO: 3 - protein, 17 aa, synthetic construct
  source 1..17: note = VH-CDR2
YISSGSFTIYYADSVKG

SEQ ID NO: 4 - protein, 9 aa, synthetic construct
  source 1..9: note = VH-CDR3
MRKGYAMDY

SEQ ID NO: 5 - protein, 16 aa, synthetic construct
  source 1..16: note = VL-CDR1
RSSQIIIHSDGNTYLE

SEQ ID NO: 6 - protein, 7 aa, synthetic construct
  source 1..7: note = VL-CDR2
KVSNRFS

SEQ ID NO: 7 - protein, 9 aa, synthetic construct
  source 1..9: note = VL-CDR3
FQGSHVPHT

SEQ ID NO: 8 - protein, 118 aa, synthetic construct
  source 1..118: note = VH
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSS

SEQ ID NO: 9 - protein, 112 aa, synthetic construct
  source 1..112: note = VL
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIK

SEQ ID NO: 10 - protein, 447 aa, synthetic construct
  source 1..447: note = Heavy chain of CD56-A
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 219 aa, synthetic construct
  source 1..219: note = Light chain of CD56-A and CD56-B
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 447 aa, synthetic construct
  source 1..447: note = Heavy chain of CD56-B
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 329 aa, synthetic construct
  source 1..329: note = IgG1 Heavy Chain Constant Region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 329 aa, synthetic construct
  source 1..329: note = IgG1 Heavy Chain Constant Region S239C
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 329 aa, synthetic construct
  source 1..329: note = IgG1 Heavy Chain Constant Region K334C
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 327 aa, synthetic construct
  source 1..327: note = IgG4 Heavy Chain Constant Region S228P
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 329 aa, synthetic construct
  source 1..329: note = IgG1 N297A Constant regions (CH1-Hinge-CH2-CH3)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 329 aa, synthetic construct
  source 1..329: note = IgG1 L234A, L235A Constant regions (CH1-Hinge-CH2-CH3)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 329 aa, synthetic construct
  source 1..329: note = IgG1 L234A, L235A, N297A Constant regions (CH1-Hinge-CH2-CH3)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG